(19) United States
(12) Patent Application Publication
(10) Pub. No.: US 2004/0009950 A1
(43) Pub. Date: Jan. 15, 2004

(54) Secreted human proteins

(75) Inventor: Pablo D. Garcia, San Francisco, CA (US)

Correspondence Address: Chiron Corporation, Intellectual Property - R440, P.O. Box 8097, Emeryville, CA, 94662-8097, US

(73) Assignee: CHIRON CORPORATION, EMERYVILLE, CA (US)

(21) Appl. No.: 10/458,143

(22) Filed: Jun. 9, 2003

Related U.S. Application Data
(63) Continuation of application No. 09/546,309, filed on Apr. 10, 2000, now abandoned.
(60) Provisional application No. 60/128,574, filed on Apr. 9, 1999.
(60) Provisional application No. 60/150,054, filed on Aug. 20, 1999.

(51) Int. Cl. A61K 48/00; A61K 38/17; C12P 21/02; C12N 5/06; C07K 14/47; C07H 21/04

(52) U.S. Cl. 514/44; 514/12; 435/69.1; 435/320.1; 435/325; 530/350; 536/23.2

(57) ABSTRACT

Fifteen secreted human proteins and full-length cDNA sequences encoding the proteins have been identified. The proteins have various potential uses as therapeutics, such as for stimulating blood cell generation in patients receiving cancer chemotherapy, for treatment of bone marrow transplantation patients, and for healing fractured bones. The proteins and cDNA sequences can also be used, inter alia, for targeting other proteins to the membrane or extracellular milieu.

### TECHNICAL AREA OF THE INVENTION

This invention relates to proteins secreted from bone marrow and from fetal liver, and to polynucleotides encoding the secreted proteins. The invention also relates to therapeutic and diagnostic utilities for the polynucleotides and proteins.

### BACKGROUND OF THE INVENTION

Human tissues, such as fetal liver and bone marrow stromal cells, secrete a variety of protein factors. Some of these factors are required for the formation of blood and bone cells and for other physiological processes. Regulatory factors which are known to be involved in hematopoiesis and/or bone development include SCF, IL-3, IL-6, GM-CSF, M-CSF, EPO, TPO, bone morphogenic proteins, erythroid potentiating factor, and TGF-β. However, it is believed that additional secreted protein factors which control hematopoiesis and bone morphogenesis remain to be identified. Other secreted proteins may play a role in cell-cell interaction and regulation of cell growth, both of which are related to cancer. There is a need to identify such proteins.

### SUMMARY OF THE INVENTION

It is an object of the invention to provide novel secreted proteins and polynucleotide sequences which encode the proteins. These and other objects of the invention are provided by one or more of the embodiments described below.

One embodiment of the invention is an isolated and purified protein having an amino acid sequence which is at least 85% identical to an amino acid sequence selected from the group consisting of the amino acid sequences shown in SEQ ID NOS:2, 4, 6, 8, 10, 12, 14, 16, 18, 20, 22, 24, 26, 28 and 30. Percent identity can be determined using a Smith-Waterman homology search algorithm using an affine gap search with a gap open penalty of 12 and a gap extension penalty of 1.

Another embodiment of the invention is an isolated and purified polypeptide comprising at least 8 contiguous amino acids of an amino acid sequence selected from the group consisting of the amino acid sequences shown in SEQ ID NOS:2, 4, 6, 8, 10, 12, 14, 16, 18, 20, 22, 24, 26, 28 and 30.

Still another embodiment of the invention is a fusion protein comprising a first protein segment and a second protein segment fused together by means of a peptide bond. The first protein segment consists of at least 8 contiguous amino acids of an amino acid sequence selected from the group consisting of the amino acid sequences shown in SEQ ID NOS:2, 4, 6, 8, 10, 12, 14, 16, 18, 20, 22, 24, 26, 28 and 30.

Yet another embodiment of the invention is a preparation of antibodies which specifically bind to a protein having an amino acid sequence selected from the group consisting of the amino acid sequences shown in SEQ ID NOS:2, 4, 6, 8, 10, 12, 14, 16, 18, 20, 22, 24, 26, 28 and 30.

Even another embodiment of the invention is a cDNA molecule which encodes a protein having an amino acid sequence which is at least 85% identical to an amino acid sequence selected from the group consisting of the amino acid sequences shown in SEQ ID NOS:2, 4, 6, 8, 10, 12, 14, 16, 18, 20, 22, 24, 26, 28 and 30. Percent identity is determined using a Smith-Waterman homology search algorithm using an affine gap search with a gap open penalty of 12 and a gap extension penalty of 1.

A further embodiment of the invention is a cDNA molecule which encodes at least 8 contiguous amino acids of an amino acid sequence selected from the group consisting of the amino acid sequences shown in SEQ ID NOS:2, 4, 6, 8, 10, 12, 14, 16, 18, 20, 22, 24, 26, 28 and 30.

Another embodiment of the invention is a cDNA molecule comprising a nucleotide sequence selected from the group consisting of at least 69 contiguous nucleotides of SEQ ID NO:1, at least 550 contiguous nucleotides of SEQ ID NO:3, at least 180 contiguous nucleotides of SEQ ID NO:5; at least 27 contiguous nucleotides of SEQ ID NO:7, and at least 11 contiguous nucleotides of SEQ ID NO:9.

Still another embodiment of the invention is a cDNA molecule which is at least 85% identical to a nucleotide sequence selected from the group consisting of the nucleotide sequences shown in SEQ ID NOS:1, 3, 5, 7, 9, 11, 13, 15, 17, 19, 21, 23, 25, 27 and 29. Percent identity is determined using a Smith-Waterman homology search algorithm using an affine gap search with a gap open penalty of 12 and a gap extension penalty of 1.

Even another embodiment of the invention is an isolated and purified polynucleotide molecule comprising a nucleotide sequence which hybridizes to a nucleotide sequence selected from the group consisting of the nucleotide sequences shown in SEQ ID NOS:1, 3, 5, 7, 9, 11, 13, 15, 17, 19, 21, 23, 25, 27 and 29 after washing with 0.2× SSC at 65° C. The nucleotide sequence encodes a protein having an amino acid sequence selected from the group consisting of the amino acid sequences shown in SEQ ID NOS:2, 4, 6, 8, 10, 12, 14, 16, 18, 20, 22, 24, 26, 28 and 30.

Yet another embodiment of the invention is a polynucleotide construct comprising a promoter and a polynucleotide segment encoding at least 8 contiguous amino acids of a protein as shown in SEQ ID NOS:2, 4, 6, 8, or 10. The polynucleotide segment is located downstream from the promoter. Transcription of the polynucleotide segment initiates at the promoter.

A further embodiment of the invention is a host cell comprising a polynucleotide construct. The polynucleotide construct comprises a promoter and a polynucleotide segment encoding at least 8 contiguous amino acids of a protein as shown in SEQ ID NOS:2, 4, 6, 8, or 10. The polynucleotide segment is located downstream from the promoter. Transcription of the polynucleotide segment initiates at the promoter.

Even another embodiment of the invention is a method of producing a human protein. A host cell comprising a polynucleotide construct is cultured in a culture medium. The polynucleotide construct comprises a promoter and a polynucleotide segment encoding at least 8 contiguous amino acids of a protein as shown in SEQ ID NOS:2, 4, 6, 8, or 10. The polynucleotide segment is located downstream from the promoter. Transcription of the polynucleotide segment initiates at the promoter. The human protein is purified from the cell or the culture medium.

The present invention thus provides the art with the amino acid sequences of fifteen full-length novel human secreted proteins and with polynucleotide molecules which encode these proteins. The invention can be used to, inter alia, to produce secreted proteins for therapeutic and diagnostic purposes.

### DETAILED DESCRIPTION OF THE INVENTION

Fifteen cDNA clones have been identified which encode novel human secreted proteins. One cDNA clone (ch1268) contains a 1313 basepair insert (SEQ ID NO:1) that encodes a polypeptide of 325 amino acids (SEQ ID NO:2). The open reading frame encoding this polypeptide is located from nucleotides 163 to 1137 of SEQ ID NO:1. Amino acids 1 to 19 of SEQ ID NO:2 form a cleavable signal peptide.

Another cDNA clone (ch1256) contains a 1941 basepair insert (SEQ ID NO:3) that encodes a polypeptide of 435 amino acids (SEQ ID NO:4). The open reading frame encoding this polypeptide is located from nucleotides 262 to nucleotide 1566 of SEQ ID NO:3. Amino acids 1 to 24 of SEQ ID NO:4 form a cleavable signal peptide.

Yet another cDNA clone (ch1284) contains a 1839 basepair insert (SEQ ID NO:5) that encodes a polypeptide of 339 amino acids (SEQ ID NO:6). The open reading frame encoding this polypeptide is located from nucleotides 40 to 1056 of SEQ ID NO:5. Amino acids 1 to 25 of SEQ ID NO:6 form a cleavable signal peptide.

Even another cDNA clone (ch1297) contains a 1831 basepair insert (SEQ ID NO:7) that encodes a polypeptide of 399 amino acids (SEQ ID NO:8). The open reading frame encoding this polypeptide is located from nucleotides 90 to 1286 of SEQ ID NO:7. Amino acids 1-19 of SEQ ID NO:8 form a cleavable signal peptide.

Still another cDNA clone (ch1233) contains a 4222 basepair insert (SEQ ID NO:9) that encodes a polypeptide of 709 amino acids (SEQ ID NO:10). The open reading frame encoding this polypeptide is located from nucleotides 238 to 2367 of SEQ ID NO:9. The open reading frame does not encode a cleavable signal peptide.

Another cDNA clone (ch 050) contains a 960 base pair inserts (SEQ ID NO:11) that encodes a polypeptide of 240 amino acids (SEQ ID NO:12). The open reading frame encoding this polypeptide is located from nucleotide 78 to 798. Amino acids 20 to 40 of the polypeptide contain a potential non-cleavable signal peptide and/or a transmembrane domain.

A further cDNA clone (ch1001) contains a 2832 bp insert (SEQ ID NO:13) that encodes a polypeptide of 613 amino acids (SEQ ID NO:14). The open reading frame encoding this polypeptide is located from nucleotide 317 to 2155. Amino acids 1 to 23 of the polypeptide contain a cleavable signal peptide.

Yet another cDNA clone (ch1007) contains a 3030 bp insert (SEQ ID NO:15) that encodes a polypeptide of 285 amino acids (SEQ ID NO:16). The open reading frame encoding this polypeptide is located from nucleotide 31 to 885. Amino acids 1 to 24 of the polypeptide contain a cleavable signal peptide.

Another cDNA clone (ch1035) contains a 2133 bp insert (SEQ ID NO:17) that encodes a polypeptide of 483 amino acids (SEQ ID NO:18). The open reading frame encoding this polypeptide is located from nucleotide 185 to 1633. Amino acids 1 to 20 of the polypeptide contain a cleavable signal peptide.

Still another cDNA clone (ch1063) contains a 1590 bp insert (SEQ ID NO:19) that encodes a polypeptide of 289 amino acids (SEQ ID NO:20). The open reading frame encoding this polypeptide is located from nucleotide 100 to 966. Amino acids 1 to 22 of the polypeptide contain a cleavable signal peptide.

Another cDNA clone (ch1572) contains a 1994 bp insert (SEQ ID NO:21) that encodes a polypeptide of 585 amino acids (SEQ ID NO:22). The open reading frame is located from nucleotides 132 to 1886. A hydrophobic stretch is found at positions 14 to 33, which can act as a signal sequence, and is followed by a potential signal peptidase cleavage site between amino acids 33 and 34.

Yet another cDNA clone (ch1569) contains a 1340 bp insert (SEQ ID NO:23) that encodes a polypeptide of 280 amino acids (SEQ ID NO:24). The open reading frame is located from nucleotide 79 to 919. Hydrophobic stretches are located at positions 1 to 20 and 180 to 206.

A further cDNA clone (ch1570) contains a 1011 bp insert (SEQ ID NO:25) that encodes a polypeptide of 286 amino acids (SEQ ID NO:26). The open reading frame is located from nucleotide 128 to 986. Hydrophobic stretches are found at amino acids 27 to 53, 61 to 86, 96 to 118, 206 to 246, and 257 to 279.

A still further cDNA clone (ch1529) contains a 2027 bp insert (SEQ ID NO:27) that encodes a polypeptide of 340 amino acids (SEQ ID NO:28). The open reading frame is located from nucleotide 270 to 1284. Hydrophobic stretches are found at amino acids 19 to 44, 144 to 164, 180 to 223, 231 to 255, and 260 to 280.

A further cDNA clone (ch1515) contains a 2390 bp insert (SEQ ID NO:29) that encodes a polypeptide of 347 amino acids (SEQ ID NO:30). A hydrophobic stretch of 30 amino acids is found at amino acid positions 55 to 85

The present invention provides both full-length and mature forms of the disclosed proteins. Full-length forms of the proteins have the amino acid sequences shown in SEQ ID NOS:2, 4, 6, 8, 10, 12, 14, 16, 18, 20, 22, 24, 26, 28 and 30. In the case of proteins which are membrane-bound, such as cell surface receptor proteins, soluble forms of the proteins can be obtained by deleting the nucleotide sequences which encode part or all of the intracellular and transmembrane domains of the protein and expressing a fully secreted form of the protein in a host cell. For example, the full-length forms of the proteins can be processed enzymatically to remove the signal sequence, resulting in mature forms of the proteins.

Other domains with predicted functions can also be identified. For example, transmembrane domains can be identified by examination of the amino acid sequences disclosed herein. A transmembrane domain typically contains a long stretch of 15-30 hydrophobic amino acids. Techniques for identifying intracellular and transmembrane domains, such as homology searches, can be used to identify such domains in proteins of the invention using amino acid and polynucleotide sequences disclosed herein.

Secreted proteins of the invention have a variety of uses. For example, the proteins can be used in assays to determine biological activities, such as cytokine, cell proliferation, or cellular differentiation activities, tissue growth or regeneration, activin or inhibin activity, chemotactic or chemokinetic activity, hemostatic or thrombolytic activity, receptor/ligand activity, tumor inhibition, or anti-inflammatory activity. Assays for these activities are known in the art, as disclosed below.

Proteins of the invention can also be used as biomarkers, to identify tissues or cell types which express the proteins, or to identify a stage- or disease-specific alteration in protein expression. Proteins of the invention can be used in protein interaction assays, to identify ligands or binding proteins. Compounds which affect the biological activities of the secreted proteins or their ability to interact with specific ligands can be identified using proteins of the invention in screening assays, such as the yeast two-hybrid assay. Proteins and antibodies of the invention can also be used to design diagnostic tests and therapeutic compositions for diseases which may be associated with altered expression of these proteins.

Polynucleotide molecules which encode the proteins disclosed herein can be used to propagate additional copies of the polynucleotides or to express proteins, polypeptides, or fusion proteins of the invention. The polynucleotide molecules disclosed herein can also be used, for example, as biomarkers for tissues or chromosomes, as molecular weight markers for DNA gels, to elicit immune responses, such as the formation of antibodies against single- or double-stranded DNA, and in DNA-ligand interaction assays, to detect proteins or other molecules which interact with the polynucleotide sequences.

Disease states may be associated with alterations in the expression of genes which encode proteins of the invention. Polynucleotide sequences disclosed herein can thus be used to determine the involvement of any of these sequences in disease states. For example, a gene in a diseased cell can be sequenced and compared with a wild-type coding sequence of the invention. Alternatively, nucleotide probes can be constructed and used to detect normal or mutant forms of mRNA in a diseased cell. Polynucleotide molecules of the invention can also be used to design diagnostic tests and therapeutic compositions for diseases which may be associated with altered expression of these genes.

### Polypeptide Fragments

The invention provides polypeptide fragments of each of the disclosed proteins. Polypeptide fragments of the invention can comprise at least 8, 10, 12, 15, 18, 19, 20, 25, 50, 75, 100, 125, 130, 135, 140, 145, 150, 200, 250, 300, or 320 contiguous amino acids selected from SEQ ID NO:2. One preferred polypeptide fragment comprises amino acids 1-19 of SEQ ID NO:2.

Other polypeptide fragments can comprise at least 8, 10, 12, 15, 20, 24, 25, 50, 75, 100, 150, 200, 250, 300, 350, 400, or 430 contiguous amino acids of SEQ ID NO:4. A preferred polypeptide fragment comprises amino acids 1-24 of SEQ ID NO:4.

Still other polypeptide fragments can comprise at least 8, 10, 12, 15, 20, 25, 30, 50, 75, 100, 150, 200, 250, 300, or 330 contiguous amino acids of SEQ ID NO:6. A preferred polypeptide fragment comprises amino acids 1-25 of SEQ ID NO:6.

Even other polypeptide fragments can comprise at least 8, 10, 12, 15, 19, 20, 25, 30, 50, 75, 100, 150, 200, 250, 300, 350, or 375 contiguous amino acids of SEQ ID NO:8. A preferred polypeptide fragment comprises amino acids 1-19 of SEQ ID NO:8.

Yet other polypeptide fragments can comprise at least 8, 10, 12, 15, 20, 25, 30, 50, 52, 73, 75, 100, 150, 175, 180, 190, 200, 230, or 231 contiguous amino acids selected from amino acids 1-53, 137-210, 291-521, or 516-709 of SEQ ID NO:10, or at least 15, 16, 17, 18, 20, 25, 30, 35, 40, 45, 50, 55, 60, 65, 70, 75, 80, 85, or 90 contiguous amino acids selected from amino acids 45-145 of SEQ ID NO:10, or at least 8, 10, 12, 15, 20, 25, 30, 50, 75, 100, 150, 250, 300, 350, 400, 450, 500, 550, 600, 650, or 700 contiguous amino acids of SEQ ID NO:10.

Other polypeptide fragments can comprise at least 8, 10, 12, 15, 18, 19, 20, 25, 50, 75, 100, 125, 130, 140, 145, 150, 200, 250, 300, 350, 400, 450, 500, 550, or 580 contiguous amino acids of SEQ ID NO:22. Preferred fragments comprise amino acids 14-33 and amino acids 34-585.

Yet other polypeptide fragments can comprise at least 8, 10, 12, 15, 18, 19, 20, 25, 50, 75, 100, 125, 130, 140, 145, 150, 200, 250, and 275 contiguous amino acids of SEQ ID NO:24. Preferred fragments comprise amino acids 1-20; amino acids 21-280; and amino acids 180-206.

Other polypeptide fragments can comprise at least 8, 10, 12, 15, 18, 19, 20, 25, 50, 75, 100, 125, 130, 140, 145, 150, 200, 250, 275, and 280 contiguous amino acids of SEQ ID NO:26. Preferred fragments comprise amino acids 27-53; 62-86; 96-118; 206-246; and 257-279.

Further polypeptide fragments can comprise at least 8, 10, 12, 15, 18, 19, 20, 25, 50, 75, 100, 125, 130, 140, 145, 150, 200, 250, 300, 325, or 330 contiguous amino acids of SEQ ID NO:28. Preferred fragments comprise amino acids 19-44; 144-164; 180-223; 231-255; and 260-280.

Other preferred fragments can comprise at least 8, 10, 12, 15, 18, 19, 20, 25, 50, 75, 100, 125, 130, 140, 145, 150, 200, 250, 300, 325, 340 or 345 contiguous amino acids of SEQ ID NO:30. A preferred fragment comprises amino acids 55-85.

### Biologically Active Variants

Variants of the secreted proteins and polypeptides disclosed herein can also occur. Variants can be naturally or non-naturally occurring. Naturally occurring variants are found in humans or other species and comprise amino acid sequences which are substantially identical to the amino acid sequences shown in SEQ ID NOS:2, 4, 6, 8, 10, 12, 14, 16, 18, 20, 22, 24, 26, 28 and 30. Species homologs of the secreted proteins can be obtained using subgenomic polynucleotides of the invention, as described below, to make suitable probes or primers to screening cDNA expression libraries from other species, such as mice, monkeys, yeast, or bacteria, identifying cDNAs which encode homologs of the secreted proteins, and expressing the cDNAs as is known in the art.

Non-naturally occurring variants which retain substantially the same biological activities as naturally occurring protein variants, such as cytokine, cell proliferation, or cellular differentiation activities, tissue growth or regeneration, activin or inhibin activity, chemotactic or chemokinetic activity, hemostatic or thrombolytic activity, receptor/ligand activity, tumor inhibition, or anti-inflammatory activity, are also included here. Preferably, naturally or non-naturally occurring variants have amino acid sequences which are at least 85%, 90%, or 95% identical to the amino acid sequences shown in SEQ ID NOS:2, 4, 6, 8, 10, 12, 14, 16, 18, 20, 22, 24, 26, 28 and 30. More preferably, the molecules are at least 98% or 99% identical. Percent identity is determined using the Smith-Waterman homology search algorithm using an affine gap search with a gap open penalty of 12 and a gap extension penalty of 1. The Smith-Waterman homology search algorithm is taught in Smith and Waterman, Adv. Appl. Math. (1981) 2:482-489.

Guidance in determining which amino acid residues can be substituted, inserted, or deleted without abolishing biological or immunological activity can be found using computer programs well known in the art, such as DNASTAR software. Preferably, amino acid changes in secreted-protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Whether an amino acid change results in a functional secreted protein or polypeptide can readily be determined by testing the altered protein or polypeptide in a functional assay, for example, as disclosed in U.S. Pat. No. 5,654,173 and described in detail below.

Variants of the secreted proteins disclosed herein include glycosylated forms, aggregative conjugates with other molecules, and covalent conjugates with unrelated chemical moieties. Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

Preferably, amino acid changes in the secreted protein or polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of secreted protein or polypeptide variants are of the same type as a secreted protein or polypeptide comprising amino acid sequences encoded by the nucleotide sequence shown in SEQ ID NO:1, 3, 5, 7, 9, 11, 13, 15, 17, 19, 21, 23, 25, 27, and 29, although the properties and functions of variants can differ in degree. Whether an amino acid change results in a secreted protein or polypeptide variant with the appropriate differential expression pattern can readily be determined. For example, nucleotide probes can be selected from the marker gene sequences disclosed herein and used to detect corresponding mRNA in Northern blots or in tissue sections, as is known in the art. Alternatively, antibodies which specifically bind to protein products of genes can be used to detect expression of secreted proteins or variants thereof.

Secreted protein variants include glycosylated forms, aggregative conjugates with other molecules, and covalent conjugates with unrelated chemical moieties. Secreted protein variants also include allelic variants, species variants, and muteins. Truncations or deletions of regions which do not affect the differential expression of the secreted protein genes are also variants. Covalent variants can be prepared by linking functionalities to groups which are found in the amino acid chain or at the N- or C-terminal residue, as is known in the art.

It will be recognized in the art that some amino acid sequence of the polypeptide of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the disclosed polypeptide which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding, or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

Non-limiting examples of amino acid substitutions include substituting the amino acids at one or both of positions 33 and 34 of SEQ ID NO:22, thereby eliminating the potential signal peptidase cleavage site; and substituting one or more of the amino acids at positions 8, 130, 134, 145 and 151 of SEQ ID NO:26; positions 39, 56, 62, 102 and 107 of SEQ ID NO;28; and positions 147, 155 and 237 of SEQ ID NO:30, thereby preventing N-glycosylation at the substituted site(s).

### Fusion Proteins

Fusion proteins comprising proteins or polypeptide fragments of the invention also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of one or more of the disclosed proteins can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can be selected from the amino acid sequences shown in SEQ ID NOS:2, 4, 6, 8, 10, 12, 14, 16, 18, 20, 22, 24, 26, 28 and 30 or from biologically active variants of those sequences, such as those described above. The first protein segment can consist of a full-length secreted protein.

Other first protein segments can consist of at least 8, 10, 12, 15, 18, 19, 20, 25, 50, 75, 100, 125, 130, 135, 140, 145, 150, 200, 250, 300, or 320 contiguous amino acids selected from SEQ ID NO:2 or at least amino acids 1-19 of SEQ ID NO:2.

Still other first protein segments can consist of at least 8, 10, 12, 15, 20, 24, 25, 50, 75, 100, 150, 200, 250, 300, 350, 400, or 430 contiguous amino acids of SEQ ID NO:4 or at least amino acids 1-24 of SEQ ID NO:4.

Yet other first protein segments can consist of at least 8, 10, 12, 15, 20, 25, 30, 50, 75, 100, 150, 200, 250, 300, or 330 contiguous amino acids of SEQ ID NO:6 or at least amino acids 1-25 of SEQ ID NO:6.

Even other first protein segments can consist of at least 8, 10, 12, 15, 19, 20, 25, 30, 50, 75, 100, 150, 200, 250, 300, 350, or 375 contiguous amino acids of SEQ ID NO:8 or at least amino acids 1-19 of SEQ ID NO:8.

Other first protein segments can consist of at least 8, 10, 12, 15, 20, 25, 30, 50, 52, 73, 75, 100, 150, 175, 180, 190, 200, 230, or 231 contiguous amino acids selected from amino acids 1-53, 137-210, 291-521, or 516-709 of SEQ ID NO:10, at least 15, 16, 17, 18, 20, 25, 30, 35, 40, 45, 50, 55, 60, 65, 70, 75, 80, 85, or 90 contiguous amino acids selected from amino acids 45-145 of SEQ ID NO 10, or at least 8, 10, 12, 15, 20, 25, 30, 50, 75, 100, 150, 250, 300, 350, 400, 450, 500, 550, 600, 650, or 700 contiguous amino acids of SEQ ID NO:10.

Other first protein segments can consist of at least 8, 10, 12, 15, 20, 24, 25, 50, 75, 100, 125, 130, 150, 175, 200, 225, 230, 235 or 239 contiguous amino acids of SEQ ID NO:12, at least 8, 10, 12, 15, 20, 24, 25, 50, 75, 100, 125, 150, 175, 200, 250, 300, 350, 400, 450, 500, 550, 600, 605 or 610 contiguous amino acids of SEQ ID NO:14, or at least 8, 10, 12, 15, 20, 24, 25, 50, 75, 100, 125, 150, 175, 200, 225, 250, 275 or 280 contiguous amino acids of SEQ ID NO:16.

Other first protein segments can consist of at least 8, 10, 12, 15, 20, 24, 25, 50, 75, 100, 125, 130, 150, 175, 200, 250, 275, 300, 350, 400, 425, 450, 475 or 480 contiguous amino acids of SEQ ID NO:18, or at least 8, 10, 12, 15, 20, 24, 25, 50, 75, 100, 125, 150, 200, 225, 250, 275, 280 or 285 contiguous amino acids of SEQ ID NO:20.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises coding sequences selected from SEQ ID NOS:1, 3, 5, 7, 9, 11, 13, 15, 17, 19, 21, 23, 25, 27 and 29 in proper reading frame with nucleotides encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### Isolation and Production of Secreted Proteins

Secreted proteins can be extracted from human cells, such as bone marrow, spleen, thymus, or peripheral blood lymphocytes, using standard biochemical methods. These methods include, but are not limited to, size exclusion chromatography, ammonium sulfate fractionation, ion exchange chromatography, affinity chromatography, crystallization, electrofocusing, and preparative gel electrophoresis. An isolated and purified secreted protein or polypeptide is separated from other compounds which normally associate with the protein or polypeptide in a cell, such as other proteins, carbohydrates, lipids, or subcellular organelles. A preparation of isolated and purified secreted proteins or polypeptides is at least 80% pure; preferably, the preparations are 90%, 95%, or 99% pure. Purity of the preparations can be assessed by any means known in the art. For example, the purity of a preparation can be assessed by examining electrophoretograms of protein or polypeptide preparations at several pH values and at several polyacrylamide concentrations, as is known in the art.

Proteins, fusion proteins, or polypeptides of the invention can be produced by recombinant DNA methods. For production of recombinant proteins, fusion proteins, or polypeptides, coding sequences selected from the nucleotide sequences shown in SEQ ID NOS:1, 3, 5, 7, 9, 11, 13, 15, 17, 19, 21, 23, 25, 27 and 29 can be expressed in prokaryotic or eukaryotic host cells using expression systems known in the art. These expression systems include bacterial, yeast, insect, and mammalian cells (see below).

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

Proteins or polypeptides of the invention can also be expressed in cultured host cells in a form which will facilitate purification. For example, a secreted protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequences disclosed herein can also be used to construct transgenic animals, such as cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule. Variants can be similarly produced.

### Antibodies

Isolated and purified proteins, polypeptides, variants, or fusion proteins can be used as immunogens, to obtain preparations of antibodies which specifically bind to epitopes of the disclosed proteins. The antibodies can be used, inter alia, to detect wild-type secreted protein or secreted protein complexes in human tissue and fractions thereof. The antibodies can also be used to detect the presence of mutations in a gene which result in under- or over-expression of a secreted protein of the invention or in expression of a secreted protein with altered size or electrophoretic mobility.

Any type of antibody known in the art can be generated to bind specifically to epitopes of secreted proteins of the invention. For example, preparations of polyclonal and monoclonal antibodies can be made using standard methods which are well known in the art. Single-chain antibodies can also be prepared. Single-chain antibodies which specifically bind to epitopes of the disclosed proteins can be isolated, for example, from single-chain immunoglobulin display libraries, as is known in the art. The library is “panned” against a disclosed amino acid sequence, and a number of single chain antibodies which bind with high-affinity to different epitopes of a protein of the invention can be isolated. Hayashi et al., 1995, Gene 160:129-30. Single-chain antibodies can also be constructed using a DNA amplification method, such as the polymerase chain reaction (PCR), using hybridoma cDNA as a template. Thirion et al., 1996, Eur. J. Cancer Prev. 5:507-11.

Single-chain antibodies can be mono- or bispecific, and can be bivalent or tetravalent. Construction of tetravalent, bispecific single-chain antibodies is taught, for example, in Coloma and Morrison, 1997, Nat. Biotechnol. 15:159-63. Construction of bivalent, bispecific; single-chain antibodies is taught inter alia in Mallender and Voss. 1994, J. Biol. Chem. 269:199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology. Verhaar et al., 1995, Int. J. Cancer 61:497-501; Nicholls et al., 1993, J. Immunol. Meth. 165:81-91.

Monoclonal and other antibodies can also be “humanized” in order to prevent a patient from mounting an immune response against the antibody when it is used therapeutically. Such antibodies may be sufficiently similar in sequence to human antibodies to be used directly in therapy or may require alteration of a few key residues. Sequence differences between, for example, rodent antibodies and human sequences can be minimized by replacing residues which differ from those in the human sequences, for example, by site directed mutagenesis of individual residues, or by grafting of entire complementarily determining regions. Alternatively, one can produce humanized antibodies using recombinant methods, as described in GB2188638B. Antibodies which specifically bind to secreted protein epitopes can contain antigen binding sites which are either partially or fully humanized, as disclosed in U.S. Pat. No. 5,565,332.

Rodents, such as mice and rats, can be genetically engineered to produce a large repertoire of human antibodies. Segments of human immunoglobulin loci can be introduced into the germlines of these rodents. Either miniloci, containing 1-2 VH segments, or large continuous fragments of human heavy and light immunoglobulin loci can be used. If desired, gene targeting can be used to create rodents which do not make rodent antibodies. The engineered rodents produce fully human antibodies. In particular, human monoclonal antibodies with high affinity and specificity against a wide variety of antigens, including human antigens, can be produced. Methods of producing fully human antibodies from transgenic rodents are taught, for example, in Wagner et al. Eur. J. Immunol. 24: 2672-81 (1994); Lonberg et al., Nature 368: 856 59 (1994); Green et al., Nature Genet. 7: 13-21 (1994); Jakobovits, Curr. Opin. Biotechnol. 6: 561-66 (1995); Jakobovits et al, Ann. N.Y Acad. Sci. 764: 525-35 (1995); Bruggemann & Neuberger, Immunol. Today 17: 391-97 (1996); and Mendez et al., Nature Genet. 15: 146-56(1997).

Other types of antibodies can be constructed and used therapeutically. For example, chimeric antibodies can be constructed as disclosed in WO 93/03151. Binding proteins which are derived from immunoglobulins and which are multivalent and multispecific, such as the “diabodies” described in WO 94/13804, can also be prepared.

Secreted protein-specific antibodies specifically bind to epitopes present in a full-length secreted protein having an amino acid sequence as shown in SEQ ID NOS:2, 4, 6, 8, 10, 12, 14, 16, 18 or 20, to polypeptide fragments, or to variants, either alone or as part of a fusion protein. Preferably, the epitopes are not present in other human proteins. Typically, at least 6, 8, 10, or 12 contiguous amino acids are required to form an epitope. However, epitopes which involve non-contiguous amino acids may require more, e.g., at least 15, 25, or 50 amino acids.

Antibodies which specifically bind to epitopes of the disclosed proteins, polypeptides, fusion proteins, or biologically active variants can be used in immunochemical assays, including but not limited to Western blots, ELISAs, radioimmunoassays, immunohistochemical assays, immunoprecipitations, or other immunochemical assays known in the art. Typically, antibodies of the invention provide a detection signal at least 5-, 10-, or 20-fold higher than a detection signal provided with other proteins when used in such immunochemical assays. Preferably, antibodies which specifically bind to epitopes of the disclosed proteins do not detect other proteins in immunochemical assays and can immunoprecipitate a secreted protein or polypeptide of the invention from solution.

Antibodies can be purified by methods well known in the art. Preferably, the antibodies are affinity purified, by passing the antibodies over a column to which a protein, polypeptide, variant, or fusion protein of the invention is bound. The bound antibodies can then be eluted from the column, for example, using a buffer with a high salt concentration.

### Polynucleotide Sequences

Genes which encode the secreted proteins of the invention have the coding sequences shown in SEQ ID NOS:1, 3, 5, 7, 9, 11, 13, 15, 17, 19, 21, 23, 25, 27 and 29. Polynucleotide molecules of the invention contain less than a whole chromosome and can be single- or double-stranded. Preferably, the polynucleotide molecules are intron-free. Polynucleotide molecules of the invention can comprise at least 11, 15, 18, 21, 30, 33, 42, 54, 60, 66, 72, 84, 90, 10, 120, 140, 160, 180, 200, 240, 300, 330, 400, 420, 500, 540, 600, 660, 700, 720, 800, 840, 900, 960, 1000, 1100, or 1200 or more contiguous nucleotides selected from nucleotides 109-1313 of SEQ ID NO:1, at least 37, 42, 54, 60, 66, 72, 84, 90, 10, 120, 140; 160, 180, 200, 240, 300, 330, 400, 420, 500, 540, 600, 660, 700, 720, 800, 840, 900, 960, 1000, 1100, 1200, or 1230 contiguous nucleotides selected from nucleotides 84 to 1313 of SEQ ID NO:1, at least 69, 72, 84, 90, 10, 120, 140, 160, 180, 200, 240, 300, 330, 400, 420, 500, 540, 600, 660, 700, 720, 800, 840, 900, 960, 1000, 1100, 1200, 1250, or 1300 contiguous nucleotides selected from SEQ ID NO:1, the 1313 contiguous nucleotides of SEQ ID NO:1, or the complements thereof.

Other polynucleotide molecules of the invention can comprise at least 11, 15, 18, 21, 30, 33, 42, 54, 60, 66, 72, 84, 90, 10, 120, 140, 160, 180, 200, 240, 300, 330, 400, 420, 500, 540, 600, 660, 700, 720, or 800 contiguous nucleotides selected from nucleotides 1-818 of SEQ ID NO:3, at least 11, 15, 18, 21, 30, 33, 42, 54, 60, 66, 72, 84, 90, 100, 120, 140, 160, or 180 contiguous nucleotides selected from nucleotides 1762-1941 of SEQ ID NO:3 at least 550, 600, 660, 700, 720, 800, 840, 900, 960, 1000, 1100, 1200, 1250, 1295, 1300, 1350, 1400, or 1411 contiguous nucleotides selected from SEQ ID NO:3, at least 30, 33, 42, 54, 60, 66, 72, 84, 90, 10, 120, 140, 160, 180, 200, 240, 300, 330, 400, 420, 500, 550, 600, 660, 700, 720, 800, 840, 900, 960, 1000, 1100, 1200, 1250, 1295, 1300, 1350 , 1400, or 1420 contiguous nucleotides selected from nucleotides 1-1425 of SEQ ID NO:3, at least 68, 72, 84, 90, 10, 120, 140, 160, 180, 200, 240, or 300 contiguous nucleotides selected from nucleotides 1637-1941 of SEQ ID NO:3, at least 97, 100, 120, 140, 160, 180, 200, 250, 300, 350, 400, 450, 500, 550, 600, 660, 700, 720, 800, 840, 900, 960, 1000, 1100, 1200, 1250, 1295, 1300, 1350, 1400, 1450, 1500, 1550, 1600, or 1650 contiguous nucleotides selected from nucleotides 1-1652 of SEQ ID NO:3, at least 97, 100, 120, 140, 160, 180, 200, 250, 300, 350, 400, 450, 500, 550, 600, 660, 700, 720, 800, 840, 900, 960, 1000, 1100, or 1200 contiguous nucleotides selected from nucleotides 262-1556 of SEQ ID NO:3, the 1941 contiguous nucleotides of SEQ ID NO:3, or the complements thereof.

Still other polynucleotide molecules of the invention can comprise at least 11 contiguous nucleotides selected from nucleotides molecules 1-32 of SEQ ID NO:5, at least 11, 15, 18, 21, 30, 33, 42, 54, 60, 66; 72, 84, 90, 10, 120, 140, 160, 180, 200, 240, 300, 330, 400, 420, 500, 540, 600, or 640 contiguous nucleotides selected from nucleotides 191-1839 of SEQ ID NO:5, at least 180, 200, 250, 300, 350, 400, 450, 500, 550, 600, 660, 700, 720, 800, 840, 900, 960, 1000, 1017, 1100, 1200, 1250, 1295, 1300, 1350, 1400, 1450, 1500, 1550, 1600, 1650, 1700, 1750, or 1800 contiguous nucleotides selected from SEQ ID NO:5, the 1839 contiguous nucleotides of SEQ ID NO:5, or the complements thereof.

Even other polynucleotide molecules of the invention can comprise at least 27, 30, 33, 42, 54, 57, 60, 66, 72, 84, 90, 10, 120, 140, 160, 180, 200, 240, 300, 330, 400, 420, 500, 540, 600, 700, 720, 800, 840, 900, 960, 1000, 1017, 1100, 1200, 1250, 1295, 1300, 1350, 1400, 1450, 1500, 1550, 1600, 1650, 1700, 1750, or 1800 contiguous nucleotides selected from SEQ ID NO:7, at least 11, 15, 18, 21, 30, 33, 42, 54, 57, 60, 66, 72, 84, 90, 10, 120, 140, 160, 180, 200, 240, 300, 330, 400, 420, 500, 540, 600, 700, 720, 800, 840, 900, 960, 1000, 1017, 1100, 1197, 1200, 1250, 1295, 1300, 1350, 1400, 1450, 1500, 1550, 1600, 1650, 1700, 1750, or 1800 contiguous nucleotides selected from nucleotides 16-1831 of SEQ ID NO:7, the 1831 contiguous nucleotides of SEQ ID NO:7, or the complements thereof.

Other polynucleotide molecules of the invention can comprise at least 11, 15, 18, 21, 30, 33, 42, 54, 57, 60, 66, 72, 84, 90, 10, 120, 140, 160, 180, 200, 240, 300, 330, 400, 420, 500, 540, 600, 700, 720, 800, 840, 900, 960, 1000, 1017, 1100, 1197, 1200, 1250, 1295, 1300, 1350, 1400, 1450, 1500, 1550, 1600, 1650, 1700, 1750, 1800, 1850, 1900, 1950, 2000, 2100, 2200, 2300, 2400, 2500, 2600, 2700, 2800, 2900, 3000, 3100, 3200, 3300, 3400, 3500, 3600, 3700, 3800, 3900, 4000, or 4220 contiguous nucleotides selected from SEQ ID NO:9, the 4222 contiguous nucleotides of SEQ ID NO:9, or the complements thereof.

The complements of the nucleotide sequences shown in SEQ ID NOS:1, 3, 5, 7, 9, 11, 13, 15, 17, 19, 21, 23, 25, 27 and 29 are contiguous nucleotide sequences which form Watson-Crick base pairs with a contiguous nucleotide sequence as shown in SEQ ID NOS: 1, 3, 5, 7, 9, 11, 13, 15, 17, 19, 21, 23, 25, 27 and 29. The complements of the nucleotide sequences shown in SEQ ID NOS: 1, 3, 5, 7, 9, 11, 13, 15, 17, 19, 21, 23, 25, 27 and 29 (the antisense strand) can be used provide antisense oligonucleotides. Polynucleotide molecules of the invention also include molecules which encode single-chain antibodies which specifically bind to the disclosed proteins, ribozymes which specifically bind to mRNA encoding the disclosed proteins, and fusion proteins comprising amino acid sequences of the disclosed proteins.

Degenerate polynucleotide sequences which encode amino acid sequences of the secreted proteins and variants, as well as homologous nucleotide sequences which are at least 65%, 75%, 85%, 90%, 95%, 98%, or 99% identical to the nucleotide sequences shown in SEQ ID NOS: 1, 3, 5, 7, 9, 11, 13, 15, 17, 19, 21, 23, 25, 27 and 29 are also polynucleotide molecules of the invention. Percent sequence identity is determined by any method known in the art, for example, using computer programs which employ the Smith-Waterman algorithm, such as the MPSRCH program (Oxford Molecular), using an affine gap search with the following parameters: a gap open penalty of 12 and a gap extension penalty of 1.

Typically, homologous polynucleotide sequences can be confirmed by hybridization under stringent conditions, as is known in the art. For example, using the following wash conditions: 2× SSC (0.3 M NaCl, 0.03 M sodium citrate, pH 7.0), 0.1% SDS, room temperature twice, 30 minutes each; then 2× SSC, 0.1% SDS, 50° C. once, 30 minutes; then 2× SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

Species homologs of polynucleotide molecules which encode proteins of the invention can be identified by making suitable probes or primers and screening cDNA expression libraries from other species, such as mice, monkeys, yeast, or bacteria, as well as human cDNA expression libraries. It is well known that the Tm of a double-stranded DNA decreases by 1-1.5° C. with every 1% decrease in homology (Bonner et al., J. Mol. Biol. 81, 123 (1973). Homologous human polynucleotides or polynucleotides of other species can therefore be identified, for example, by hybridizing a putative homologous polynucleotide with a polynucleotide having the nucleotide sequence of SEQ ID NO:1, 3, 5, 7, 9, 11, 13, 15, 17, 19, 21, 23, 25, 27 and 29 to form a test hybrid, comparing the melting temperature of the test hybrid with the melting temperature of a hybrid of a polynucleotide consisting of a nucleotide sequence of SEQ ID NO:1, 3, 5, 7, 9, 11, 13, 15, 17, 19, 21, 23, 25, 27 and 29 and a perfectly complementary polynucleotide, and calculating the number or percent of basepair mismatches within the test hybrid.

Nucleotide sequences which hybridize to the coding sequences shown in SEQ ID NOS: 1, 3, 5, 7, 9, 11, 13, 15, 17, 19, 21, 23, 25, 27 and 29 or their complements following stringent hybridization and/or wash conditions are also polynucleotide molecules of the invention. Stringent wash conditions are well known and understood in the art and are disclosed, for example, in Sambrook et al., Molecular Cloning A Laboratory Manual, 2d ed., 1989, at pages 9.50-9.51.

Typically, for stringent hybridization conditions a combination of temperature and salt concentration should be chosen that is approximately 12-20° C. below the calculated Tm of the hybrid under study. The Tm of a hybrid between a nucleotide sequence as shown in SEQ ID NOS:1, 3, 5, 7, 9, 11, 13, 15, 17, 19, 21, 23, 25, 27 and 29 and a polynucleotide sequence which is 65%, 75%, 85%, 90%, 95%, 96%, 97%, 98%, or 99% identical can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(%G+C)−0.63(%formamide)−600/l),

where l=the length of the hybrid in basepairs.

Stringent wash conditions include, for example, 4× SSC at 65° C., or 50% formamide, 4× SSC at 42° C., or 0.5× SSC, 0.1% SDS at 65° C. Highly stringent wash conditions include, for example, 0.2× SSC at 65° C.

Polynucleotide molecules of the invention can be isolated and purified free from other nucleotide sequences using standard nucleic acid purification techniques. For example, restriction enzymes and probes can be used to isolate polynucleotide fragments which comprise nucleotide sequences encoding one or more of the secreted proteins disclosed herein. Isolated and purified polynucleotide molecules are in preparations which are free or at least 90% free of other molecules.

Complementary DNA (cDNA) molecules which encode secreted proteins of the invention are also polynucleotide molecules of the invention. cDNA molecules can be made with standard molecular biology techniques, using mRNA as a template. cDNA molecules can thereafter be replicated using molecular biology techniques known in the art and disclosed in manuals such as Sambrook et al., 1989. An amplification technique, such as the polymerase chain reaction (PCR), can be used to obtain additional copies of polynucleotide molecules of the invention, using either human genomic DNA or cDNA as a template.

Alternatively, synthetic chemistry techniques can be used to synthesize polynucleotide molecules of the invention. The degeneracy of the genetic code allows polynucleotide molecules with alternate nucleotide sequences to be synthesized which will encode a protein having an amino acid sequence as shown in SEQ ID NOS:2, 4, 6, 8, 10, 12, 14, 16, 18, 20, 22, 24, 26, 28 or 30 or a biologically active variant of one of those proteins. All such polynucleotide molecules are within the scope of the present invention.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides selected from SEQ ID NOS:1, 3, 5, 7, 9, 11, 13, 15, 17, 19, 21, 23, 25, 27 and 29. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequences shown in SEQ ID NOS:1, 3, 5, 7, 9, 11, 13, 15, 17, 19, 21, 23, 25, 27 and 29 for use in identifying or amplifying the genes from human genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

### Polynucleotide Constructs

Polynucleotide molecules comprising the coding sequences disclosed herein can be used in a polynucleotide construct, such as a DNA or RNA construct. Polynucleotide molecules of the invention can be used, for example, in an expression construct to express all or a portion of a secreted protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

### Host Cells

An expression construct can be introduced into a host cell. The host cell comprising the expression construct can be any suitable prokaryotic or eukaryotic cell. Expression systems in bacteria include those described in Chang et al., Nature (1978) 275: 615; Goeddel et al., Nature (1979) 281: 544; Goeddel et al., Nucleic Acids Res. (1980) 8: 4057; EP 36,776; U.S. Pat. No. 4,551,433; deBoer et al., Proc. Natl. Acad. Sci. USA (1983) 80: 21-25; and Siebenlist et al., Cell (1980) 20: 269.

Expression systems in yeast include those described in Hinnen et al., Proc. Natl. Acad. Sci. USA (1978) 75: 1929; Ito et al., J. Bacteriol. (1983) 153: 163; Kurtz et al., Mol. Cell. Biol. (1986) 6: 142; Kunze et al., J. Basic Microbiol. (1985) 25: 141; Gleeson et al., J. Gen. Microbiol. (1986) 132: 3459, Roggenkamp et al., Mol. Gen. Genet. (1986) 202: 302); Das et al., J Bacteriol. (1984) 158: 1165; De Louvencourt et al., J. Bacteriol. (1983) 154: 737, Van den Berg et al., Bio/Technology (1990) 8: 135; Kunze et al., J. Basic Microbiol. (1985) 25: 141;Cregg et al., Mol. Cell. Biol. (1985) 5: 3376; U.S. Pat. No. 4,837,148; U.S. Pat. No. 4,929,555; Beach and Nurse, Nature (1981) 300: 706; Davidow et al., Curr. Genet. (1985) 1p: 380; Gaillardin et al., Curr. Genet. (1985) 10: 49; Ballance et al., Biochem. Biophys. Res. Commun. (1983) 112: 284-289; Tilburn et al., Gene (1983) 26: 205-22;, Yelton et al., Proc. Natl. Acad. Sci. USA (1984) 81: 1470-1474; Kelly and Hynes, EMBO J. (1985) 4: 475479; EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. (1988) 69: 765-776; Miller et al., Ann. Rev. Microbiol. (1988) 42: 177; Carbonell et al., Gene (1988) 73: 409; Maeda et al., Nature (1985) 315:. 592-594; Lebacq-Verheyden et al., Mol. Cell Biol. (1988) 8: 3129; Smith et al., Proc. Natl. Acad. Sci. USA (1985) 82: 8404; Miyajima et al., Gene (1987) 58: 273; and Martin et al., DNA (1988) 7:99. Numerous baculoviral strains and variants and corresponding permissive insect host cells from hosts are described in Luckow et al., Bio/Technology (1988) 6:47-55, Miller et al., in GENERIC ENGINEERING (Setlow, J. K. et al. eds.), Vol. 8 (Plenum Publishing, 1986), pp. 277-279; and Maeda et al., Nature, (1985) 315: 592-594.

Mammalian expression can be accomplished as described in Dijkema et al., EMBO J. (1985) 4: 761; Gormanetal., Proc. Natl. Acad. Sci. USA (1982b) 79: 6777; Boshart et al., Cell (1985) 41: 521; and U.S. Pat. No. 4,399,216. Other features of mammalian expression can be facilitated as described in Ham and Wallace, Meth Enz. (1979) 58: 44; Barnes and Sato, Anal. Biochem. (1980) 102: 255; U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides selected from the nucleotide sequences shown in SEQ ID NOS:1, 3, 5, 7, 9, 11, 13, 15, 17, 19, 21, 23, 25, 27 and 29. The transcription unit is located upstream to a coding sequence of the endogenous gene. The exogenous regulatory sequence directs transcription of the coding sequence of the endogenous gene.

### Functional Assays

A protein of the invention can exhibit cytokine, cell proliferation (either inducing or inhibiting), or cell differentiation (either inducing or inhibiting) activity, or can induce production of other cytokines in certain cell populations. Many protein factors discovered to date, including all known cytokines, have exhibited activity in one or more factor-dependent cell proliferation assays; hence, the assays serve as a convenient confirmation of cytokine activity. The activity of a protein of the invention can be evidenced by any one of a number of routine factor-dependent cell proliferation assays for cell lines including, 32D (a mouse IL-3-dependent lymphoblast cell line, ATCC No. CRL-11346), DA2, DA1G, T10 (a human myeloma cell line, ATCC No. CRL-9068), B9, B9/11, BaF3, MC9/G, M+(preB M+), 2E8 (a mouse IL-7-dependent Lymphoblast cell line, ATCC No. TIB-239), RB5, DA1, 123, T1165, HT2 (a mouse lymphoma cell line, ATCC No. CRL-8629), CTLL2, TF-I (a human IL-5-unresponsive Lymphoblast cell line, ATCC No. CRL-2003), Mo7e, and CMK.

Assays for T-cell or thymocyte proliferation include those described in Current Protocols in Immunology, Coligan et al., eds., Greene Publishing Associates and Wiley-Interscience (particularly chapter 3, In Vitro Assays for Mouse Lymphocyte Function 3.1-3.19; and chapter 7, Immunologic Studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J Immunol. 149:3778-3783, 1992; and Bowman et al., J. Immunol. 152:1756-1761, 1994.

Assays for cytokine production and/or proliferation of spleen cells, lymph node cells, or thymocytes include those described in Kruisbeek and Shevach, Polyclonal T Cell Stimulation, in Current Protocols in Immunology, vol. I, pp. 3.12.1-3.12.14, and Schreiber, Measurement of Mouse and Human Interleukin Gamma, in Current Protocols in Immunology vol. 1, pp. 6.8.1-6.8.8.

Assays for proliferation and differentiation of hematopoietic and lymphopoietic cells include those described in Bottomly, Measurement of Human and Murine Interleukin 2 and Interleukin 4, in Current Protocols in Immunology vol. 1, pp. 6.3.1-6.3.12; deVries et al., J Exp. Med. 173: 1205-1211, 1991; Moreau et al., Nature 336:690-692, 1988; Greenberger et al., Proc. Natl. Acad. Sci. U.S.A. 80:2931-2938, 1983; Nordan, R., Measurement of mouse and human interleukin 6, in Current Protocols in Immunology vol. 1, pp. 6.6.1-6.6.5; Smith et al., Proc. Natl. Acad. Sci. U.S.A. 83:1857-1861, 1986; Bennett et al., Measurement of Human Interleukin 11, in Current Protocols in Immunology vol. 1, pp. 6.15.1; Ciarletta et al., Measurement of mouse and human Interleukin 9, in Current Protocols in Immunology vol. 1, p. 6.13.1.

Assays for T cell clone responses to antigens (which will identify, among others, proteins that affect APC-T cell interactions as well as direct T cell effects by measuring proliferation and cytokine production) include those described in Current Protocols in Immunology especially chapters 3 (In Vitro Assays for Mouse Lymphocyte Function), chapter 6 (Cytokines and Their Cellular Receptors), and chapter 7 (Immunologic Studies in Humans); Weinberger et al., Proc. Natl. Acad. Sci. USA 77:6091-6095, 1980; Weinberger et al., Eur. J Immun. 11:405-411, 1981; Takai et al., J Immunol. 137:3494-3500, 1986; and Takai et al., J: Immunol. 140:508-512, 1988.

Assays for tissue generation activity include those described for bone, cartilage, and tendon in WO 95/16035, for neuronal tissue in WO 95/05846, and for skin and endothelial tissue in WO 91/07491. Assays for wound healing activity include, for example, those described in Winter, Epidermal Wound Healing, polypeptides 71-112 (Maibach and Rovee, eds.), Year Book Medical Publishers, Inc., Chicago, and Eaglstein and Mertz, J. Invest. Dermatol 71:382-84 (1978).

A protein of the present invention can also demonstrate activity as a receptor, receptor ligand, or inhibitor or agonist of a receptor/ligand interaction. Examples of such receptors and ligands include cytokine receptors and their ligands, receptor kinases and their ligands, receptor phosphatases and their ligands, receptors involved in cell-cell interactions and their ligands, including cellular adhesion molecules such as selecting, integrins, and their ligands, and receptor/ligand pairs involved in antigen presentation, antigen recognition and development of cellular and humoral immune responses. Receptors and ligands are also useful for screening of potential peptide or small molecule inhibitors of the relevant receptor/ligand interaction. A protein of the invention, including fragments of receptors and ligands, can itself be useful as an inhibitor of receptor/ligand interactions.

Suitable assays for receptor-ligand activity include those described in Current Protocols in Immunology, chapter 7.28, Measurement of Cellular Adhesion Under Static Conditions, pages 7.28.1-7.28.22, Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stittetal., Cell 80:661-670, 1995.

Suitable assays for proliferation and differentiation of various hematopoietic lines are cited above. Assays for embryonic stem cell differentiation which can identify proteins which influence embryonic hematopoiesis include those described in Johansson et al. Cellular Biology 15:141-151, 1995; Keller et al., Molecular and Cellular Biology 13:473-486, 1993; and McClanahan et al., Blood 81:2903-2915, 1993.

Assays for stem cell survival and differentiation include those described in Freshney, Methylcellulose colony forming assays, in Culture of Hematopoietic Cells, Freshney et al. eds., pp. 265-268, Wiley-Liss, Inc., New York, N.Y. 1994; Hirayamaet all, Proc. Natl. Acad. Sci. USA 89:5907-5911, 1992; McNiece and Briddell, Primitive hematopoietic colony forming cells with high proliferative potential, in Culture of Hematopoietic Cells, pp. 23-39; Neben et al., Experimental Hematology 22:353-359, 1994; Ploemacher, Cobblestone area forming cell assay, in Culture of Hematopoietic Cells, pp. 1-21; Spooncer et al., Long term bone marrow cultures in the presence of stromal cells, in Culture of Hematopoietic Cells, pp. 163-179; Sutherland, Long term culture initiating cell assay, in Culture of Hematopoietic Cells, pp. 139-162. Such assays can be used to identify proteins which regulate lympho-hematopoiesis.

### Therapeutic Uses of Secreted Proteins and Polynucleotides Molecules

A protein of the present invention can be used to support colony forming cells or factor-dependent cell lines, to regulate hematopoiesis, and to treat myeloid or lymphoid cell deficiencies. The protein can be used, either alone or in combination with other cytokines, to support the growth and proliferation of erythroid progenitor cells. Proteins of the invention can also be used to treat various anemias, in conjunction with irradiation or chemotherapy to stimulate the production of erythroid precursors or erythroid cells.

A protein of the invention which has CSF activity can be used to support the growth and proliferation of myeloid cells, such as granulocytes, monocytes, or macrophages. Proteins with such activity can be used, for example, in conjunction with chemotherapy to prevent or treat myelo-suppression. Proteins of the invention can also be used to support the growth and proliferation of megakaryocytes and platelets, thereby allowing prevention or treatment of platelet disorders such as thrombocytopenia. Proteins with such activity can be used to support the growth and proliferation of hematopoietic stem cells, either in place of or in conjunction with platelet transfusions. Proteins of the invention can be used to treat stem cell disorders, such as aplastic anemia and paroxysmal nocturnal hemoglobinuria, or to repopulate the stem cell compartment after irradiation or chemotherapy, either in vivo or ex vivo. For example, a protein of the invention can be used in conjunction with homologous or heterologous bone marrow transplantation or peripheral progenitor cell transplantation.

Proteins of the invention, or fragments thereof, can be useful for treatment and diagnosis of a variety of conditions in which the rate of cell growth, and cell-cell interactions, are disrupted. Such conditions include cancer.

A protein of the invention also can have utility in compositions used for growth or differentiation of bone, cartilage, tendon, ligament, or nerve tissue, as well as for wound healing and tissue repair and replacement, and in the treatment of burns, incisions, and ulcers.

Proteins of the present invention can induce cartilage and/or bone growth in circumstances where bone is not normally formed and thus have an application in healing bone fractures and cartilage damage or defects in humans and other animals. A preparation employing a protein of the invention can have prophylactic use in closed as well as open fracture reduction and also in the improved fixation of artificial joints. De novo bone formation induced by an osteogenic agent contributes to the repair of congenital, trauma- or surgery-induced craniofacial defects and also is useful in cosmetic plastic surgery.

A protein of this invention can also be used in the treatment of periodontal disease and in other tooth repair processes. Such agents can provide an environment to attract bone-forming cells, stimulate growth of bone-forming cells, or induce differentiation of progenitors of bone-forming cells. A protein of the invention can be used to treat osteoporosis or osteoarthritis, for example, through stimulation of bone and/or cartilage repair or by blocking inflammation. Mechanisms of destroying tissue mediated by inflammatory processes, such as collagenase or osteoclast activity, can also be inhibited.

Tendon or ligament formation can also be influenced by a protein of the invention. A protein of the invention which induces tendon/ligament-like tissue or other tissue formation in circumstances where such tissue is not normally formed can be used to heal tendon or ligament tears, deformities, and other tendon or ligament defects in humans and other animals. A preparation employing a tendon/ligament-like tissue inducing protein can be used to prevent damage to tendon or ligament tissue, as well as in the improved fixation of tendon or ligament to bone or other tissues, and to repair defects to tendon or ligament tissue. De novo tendon/ligament-like tissue formation induced by a composition of the invention contributes to the repair of congenital, trauma-induced, or other tendon or ligament defects of other origin and can also be used in cosmetic plastic surgery, for attachment or repair of tendons or ligaments.

A protein of the invention can also be useful for proliferation of neural cells and for regeneration of nerve and brain tissue, i.e. for the treatment of central and peripheral nervous system diseases and neuropathies, as well as mechanical and traumatic disorders. More specifically, a protein can be used in the treatment of diseases such as Alzheimer's disease, Parkinson's disease, Huntington's disease, amyotrophic lateral sclerosis, and Shy-Drager syndrome. Other conditions which can be treated in accordance with the invention include mechanical and traumatic disorders, such as spinal cord disorders and head trauma, and cerebrovascular diseases, such as stroke. Peripheral neuropathies resulting from chemotherapy or other medical therapies can be treated using a protein of the invention.

Proteins of the invention can also be used to promote better or faster closure of non-healing wounds, including pressure ulcers, ulcers associated with vascular insufficiency, or surgical and traumatic wounds.

A protein of the invention can also affect generation or regeneration of other tissues, such as organs (including, for example, pancreas, liver, intestine, kidney, skin, endothelium), muscle (smooth, skeletal, or cardiac), and vascular (including vascular endothelium) tissue, or for promoting the growth of cells of which such tissues are comprised. Part of the desired effects can be by inhibition or modulation of fibrotic scarring to allow normal tissue to regenerate. A protein of the invention can also exhibit angiogenic activity.

A protein of the present invention can be useful for gut protection or regeneration, and for treatment of lung or liver fibrosis, reperfusion injury in various tissues, and conditions resulting from systemic cytokine damage. A protein of the invention can also be useful for promoting or inhibiting differentiation of tissues described above from precursor tissues or cells or for inhibiting the growth of tissues described above.

Secreted proteins and polynucleotides of the invention can be used in a composition. Compositions of the invention relate to isolated (purified) polypeptides and polynucleotides. These compositions are substantially free of other human proteins or human polynucleotides. A composition containing A is “substantially free of” B when at least 85% by weight of the total A+B in the composition is A. Preferably, A comprises at least about 90% by weight of the total of A+B in the composition, more preferably at least about 96% or even 99% by weight.

A protein of the invention can be used in a pharmaceutical composition. Compositions comprising proteins or polynucleotides of the invention have therapeutic applications, both for human patients and veterinary patients, such as domestic animals and thoroughbred horses. Such compositions can optionally include a pharmaceutically acceptable carrier. In addition to protein and carrier, such a composition can also contain diluents, fillers, salts, buffers, stabilizers, solubilizers, and other materials well known in the art. Characteristics of a carrier will depend on the route of administration. Compositions of the invention can also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNFO, TNF1, TNF2, G-CSF, Meg-CSF, thrombopoietin, stem cell factor, erythropoietin, or growth factors such as epidermal growth factor (EGF), platelet derived growth factor (PDGF), transforming growth factors (TGF-α and TGF-β), or insulin-like growth factor (IGF).

A pharmaceutical composition can also contain other agents which either enhance the activity of the protein or complement its activity or use in treatment. Such additional factors and/or agents can be included in the pharmaceutical composition to produce a synergistic effect with a protein of the invention or to minimize side effects. Conversely, a protein of the invention can be included in formulations of a particular factor, such as a cytokine, lymphokine, other hematopoietic factor, thrombolytic or anti-thrombotic factor, or anti-inflammatory agent to minimize side effects of the factor.

A protein of the present invention can be active in multimers (e.g., heterodimers or homodimers) or complexes with itself or other proteins, and compositions of the invention can comprise a protein of the invention in such a multimeric or complexed form. For example, a composition of the invention can be in the form of a complex of a protein or proteins of the invention together with protein or peptide antigens. The protein or peptide antigen will deliver a stimulatory signal to both B and T Lymphocytes. B Lymphocytes will respond to antigen through their surface immunoglobulin receptor. T Lymphocytes will respond to antigen through the T cell receptor (TCR) following presentation of the antigen by MHC proteins. MHC proteins and structurally related proteins, including those encoded by class I and class II MHC genes on host cells, can present the peptide antigen(s) to T Lymphocytes. Antigen components could also be supplied as purified MUC-peptide complexes alone or, with co-stimulatory molecules which can directly signal T cells. Alternatively, antibodies able to bind surface immunoglobulin and other molecules on B cells, as well as antibodies able to bind the TCR and other molecules on T cells, can be combined with a composition of the invention.

A composition of the invention can be in the form of a liposome in which a protein of the invention is combined, in addition to other pharmaceutically acceptable carriers, with amphipathic agents such as lipids, which exist in aggregated form as micelles, insoluble monolayers, liquid crystals, or lamellar layers in aqueous solution. Suitable lipids for liposomal formulation include monoglycerides, diglycerides, sulfatides, lysolecithin, phospholipids, saponin, bile acids, and the like. Preparation of liposomal formulations is within the level of skill in the art, as disclosed, for example, in U.S. Pat. No. 4,235,871, U.S. Pat. No. 4,501,728, U.S. Pat. No. 4,837,028, and U.S. Pat. No. 4,737,323.

A therapeutically effective amount of a protein of the invention is administered to a mammal having a condition to be treated. The amount of protein which is therapeutically effective is that amount of protein which is sufficient to treat, heal, prevent, or ameliorate the condition, or to increase the rate of such treatment. Proteins of the invention can be administered either alone or in combination with other therapeutic agents, such as cytokines, lymphokines, or other hematopoietic factors. Other therapeutic agents can be administered simultaneously or sequentially with proteins of the invention, as determined by the attending physician.

Compositions of the invention can be inhaled, ingested, applied topically, or administered by cutaneous, subcutaneous, intraperitoneal, parenteral or intravenous injection. When a therapeutically effective amount of protein of the present invention is administered orally, protein of the present invention will be in the form of a tablet, capsule, powder, solution or elixir. When administered in tablet form, the pharmaceutical composition of the invention can additionally contain a solid carrier such as a gelatin or an adjuvant. The tablet, capsule, and powder contain from about 5-95%, 25-90%, 30-80%, 40-75%, or 50% protein of the invention by weight. When administered in liquid form, a liquid carrier such as water, petroleum, oils of animal or plant origin such as peanut oil, mineral oil, soybean oil, or sesame oil, or synthetic oils can be added.

The liquid form of the composition can further contain physiological saline solution, dextrose or other saccharide solution, or glycols such as ethylene glycol, propylene glycol, or polyethylene glycol. When administered in liquid form, the pharmaceutical composition contains from about 0.5-90%, 1-80%, 5-75%, 10-65%, 20-50%, 10-50%, or 25-40% by weight of protein of the invention.

When a therapeutically effective amount of protein of the present invention is administered by intravenous, cutaneous, or subcutaneous injection, a pyrogen-free, parenterally acceptable aqueous solution of the protein is preferred. The skilled artisan can readily prepare an acceptable protein solution with suitable pH, isotonicity, and stability. A solution of the composition for intravenous, cutaneous, or subcutaneous injection should also contain an isotonic vehicle, such as Sodium Chloride Injection, Ringer's Injection, Dextrose Injection, Dextrose and Sodium Chloride Injection, Lactated Ringer's Injection, or other vehicles as are known in the art. Stabilizers, preservatives, buffers, antioxidants, or other additives known to those of skill in the art can also be added to the composition.

The amount of protein of the present invention in the pharmaceutical composition of the present invention will depend upon the nature and severity of the condition being treated and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention can be administered until the optimal therapeutic-effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of protein of the present invention per kg body weight.

Duration of intravenous therapy using a composition of the invention will vary, depending on the severity of the disease being treated and the condition and potential idiosyncratic response of each individual patient. It is contemplated that the duration of each application of a composition of the invention will be in the range of 12 to 24 hours of continuous intravenous administration. Ultimately, the attending physician will decide on the appropriate duration of intravenous therapy.

A composition of the invention which is useful for bone, cartilage, tendon or ligament regeneration can be administered topically, systematically, or locally in an implant or device. Encapsulation or injection in a viscous form for delivery to the site of bone, cartilage or tissue damage is also possible. Topical administration can be suitable for wound healing and tissue repair. Optionally, therapeutic agents other than a protein of the invention can be included in the composition, as described above.

For affecting bone or cartilage formation, a composition of the invention would include a matrix capable of delivering the composition to the site of bone or cartilage damage and for providing a structure for the developing bone and cartilage. Optimally, the matrix would be capable of resorption into the body. Matrices can be formed of materials presently in use for other implanted medical applications, the choice of material being based on biocompatibility, biodegradability, mechanical properties, cosmetic appearance, and interface properties. Suitable biodegradable matrix materials include chemically defined calcium sulfate, tricalciumphosphate, hydroxyapatite, polylactic acid, polyglycolic acid, polyanhydride, bone or dermal collagen, pure proteins, and extracellular matrix components. Suitable nonbiodegradable and chemically defined matrix materials include sintered hydroxyapatite, bioglass, aluminates, or other ceramics. Individual matrix components can be modified, for example, to affect pore size, particle size, particle shape, and biodegradability. Combinations of materials can be used, as is known in the art.

Sequestering agents, such as carboxymethyl cellulose or an autologous blood clot, can be employed to prevent protein compositions from dissociating from the matrix. Sequestering agents include cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, polyethylene glycol, polyoxyethylene oxide, carboxyvinyl polymer and polyvinyl alcohol. The amount of sequestering agent is based on total formulation weight, such as 0.5-20% or 1-10%, and should be an amount of sequestering agent which prevents desorbtion of the protein from the polymer matrix but which permits progenitor cells to infiltrate the matrix, so that the protein can assist the osteogenic activity of the progenitor cells.

Compositions comprising proteins of the invention can provide an environment which will attract tendon- or ligament-forming cells, stimulate growth of tendon- or ligament-forming cells, induce differentiation of progenitors of tendon-or ligament-forming cells, or induce growth of tendon/ligament cells or progenitors ex vivo. Such cells can then be returned to the body to effect tissue repair. Compositions of the invention can also be used to treat tendonitis, carpal tunnel syndrome, and other tendon or ligament defects. Such compositions can optionally include an appropriate matrix and/or sequestering agent as a pharmaceutically acceptable carrier, as is well known in the art.

The dosage regimen of a protein-containing pharmaceutical composition to be used in tissue regeneration will be determined by the attending physician considering various factors which modify the action of the proteins, e.g. amount of tissue weight desired to be formed, the site of damage, the condition of the damaged tissue, the size of a wound, type of damaged tissue (e.g., bone), the patient's age, sex, and diet, the severity of any infection, time of administration, and other clinical factors. The dosage can vary with the type of matrix used in the reconstitution and whether other therapeutic agents, such as growth factors, are included. Progress of the treatment can be monitored by periodic assessment of tissue/bone growth and/or repair, for example, using X-rays, histomorphometric determinations, or tetracycline labeling.

Polynucleotides of the invention can also be used for gene therapy. Polynucleotides can be introduced either in vivo or ex vivo into cells for expression in a mammalian subject. Cells can be cultured ex vivo in the presence of proteins of the invention in order to produce a desired effect on or activity in such cells. Treated cells can then be introduced in vivo for therapeutic purposes, as is known in the art. Polynucleotides of the invention can be administered by known methods of introducing polynucleotides into a cell or organism (including in the form of viral vectors or naked DNA).

Polynucleotides of the invention can also be delivered to subjects for the purpose of screening test compounds for those which are useful for enhancing transfer of polynucleotides of the invention to a cell or for enhancing subsequent biological effects of the polynucleotides within the cell. Such biological effects include hybridization to complementary mRNA and inhibition of its translation, expression of the polynucleotide to form mRNA and/or protein, and replication and integration of the polynucleotide.

Test compounds which can be screened include any substances, whether natural products or synthetic, which can be administered to the subject. Libraries or mixtures of compounds can be tested. The compounds or substances can be those for which a pharmaceutical effect is previously known or unknown. The compounds or substances can be delivered before, after, or concomitantly with the polynucleotides. They can be administered separately or in admixture with the polynucleotides.

Integration of delivered polynucleotides can be monitored by any means known in the art. For example, Southern blotting of the delivered polynucleotides can be performed. A change in the size of the fragments of the delivered polynucleotides indicates integration. Replication of the delivered polynucleotides can be monitored inter alia by detecting incorporation of labeled nucleotides combined with hybridization to a specific nucleotide probe. Expression of a polynucleotide of the invention can be monitored by detecting production of mRNA which hybridizes to the delivered polynucleotide or by detecting protein. Proteins of the invention can be detected immunologically. Thus, delivery of polynucleotides of the invention according to the present invention provides an excellent system for screening test compounds for their ability to enhance delivery, integration, hybridization, expression, replication or integration in an animal, preferably a mammal, more preferably a human.

### Research Uses of the Polynucleotides and Secreted Proteins

Polynucleotides of the invention can be used for a variety of research purposes. Any or all of these research utilities are capable of being developed into reagent grade or kit format for commercialization as research products. For example, polynucleotides can be used to express recombinant protein for analysis, characterization, or therapeutic use. Polynucleotides can be used as markers for tissues in which the corresponding protein is preferentially expressed, either constitutively or at a particular stage of tissue differentiation or development or in disease states. Polynucleotides can also be used as molecular weight markers on Southern gels or, when labeled, for example, with a fluorescent tag or a radiolabel, polynucleotides can be used as chromosome markers, to identify chromosomes for gene mapping.

Potential genetic disorders can be identified by comparing the sequences of wild-type polynucleotides of the invention with endogenous nucleotide sequences in patients. Polynucleotides of the invention can also be used as probes for the discovery of novel, related DNA sequences, to derive PCR primers for genetic fingerprinting, as probes to “subtract-out” known sequences in the process of discovering other novel polynucleotides, for selecting and making oligomers for attachment to a gene chip or other support, to raise anti-protein antibodies using DNA immunization techniques, and as immunogens, to raise anti-DNA antibodies or to elicit another immune response.

Where the polynucleotide encodes a protein which binds or potentially binds to another protein, such as in a receptor-ligand interaction, the polynucleotide can also be used in interaction trap assays, such as the yeast two-hybrid assay, to identify polynucleotides encoding the protein with which binding occurs or to identify inhibitors of the binding interaction, for example in drug screening assays.

Proteins of the invention can similarly be used in assays to determine biological activity, including use in a panel of multiple proteins for high-throughput screening, to raise antibodies or to elicit another immune response, as a reagent in assays designed to quantitatively determine levels of the protein (or its receptor) in biological fluids, as markers for tissues in which the protein is preferentially expressed (either constitutively or at a particular stage of tissue differentiation or development or in a disease state), and to identify related receptors or ligands. Where the protein binds or potentially binds to another protein such as, for example, in a receptor-ligand interaction, the protein can be used to identify the other protein with which binding occurs or to identify inhibitors of the binding interaction. Proteins involved in these binding interactions can also be used to screen for peptide or small molecule inhibitors or agonists of the binding interaction.

### EXAMPLES

### Example 1

### Identification of Trans-Membrane Human Proteins

A cDNA clone designated ch1572 was isolated from a fetal liver library. The cDNA contained a 1994 base pair insert (SEQ ID NO:21) encoding a 585 amino acid protein (SEQ ID NO:22). The amino acid sequence contained a hydrophobic region of amino acids at positions 14 to 33, followed by a potential signal peptidase cleavage site between amino acids 33 and 34.

Five potential N-linked glycoprotein sites were identified, at amino acids 89, 106, 189, 220 and 315 of SEQ ID NO:22. When the protein was translated in the presence of endoplasmic reticulum membranes, the molecular weight increased in a manner consistent with glycosylation.

A cDNA clone designated ch1569 was isolated from a fetal liver library. The cDNA contained a 1340 base pair insert (SEQ ID NO:23) encoding a 280 amino acid protein (SEQ ID NO:24). Hydrophobic regions were found at amino acids 1 to 20 and 180 to 206, and a potential signal peptidase cleavage site was located between amino acids 20 and 21. No potential glycosylation sites were found. Where the protein was translated in the presence of rough endoplasmic reticulum, decreased molecular weights was observed, consistent with removal of the signal peptide.

A cDNA clone designated ch1570 was isolated from a fetal liver library. The cDNA contained a 1011 base pair insert (SEQ ID NO:25) encoding a 286 amino acid protein (SEQ ID NO:26). Five hydrophobic stretches were found, at positions 27 to 53, 62 to 86, 96 to 118, 206 to 246, and 257 to 279. Potential glycosylation sites were found at positions 8, 130, 134, 145, and 151. When the protein was translated in the presence of endoplasmic reticulum, the molecular weight increased, consistent with glycosylation.

A cDNA clone designated ch1529 was isolated from a fetal liver library. The cDNA contained a 2027 base pair insert (SEQ ID NO:27) encoding a 340 amino acid protein. Five hydrophobic stretches were found, at amino acid positions 19 to 44, 144 to 164, 180 to 223, 231 to 255, and260 to 280.

Potential N-linked glycosylation sites were found at positions 39, 56, 62, 102 and 107. When the protein was translated in the presence of rough endoplasmic reticulum, the molecular weight increased, consistent with glycosylation of the protein.

A cDNA clone designated ch1515 was isolated from a fetal liver library. The cDNA contained a 2390 base pair insert (SEQ ID NO:29) encoding a 347 amino acid protein (SEQ ID NO:30). The protein contained a 30 amino acid hydrophobic region between amino acids 55 to 85, which could act as a signal peptide and/or a transmembrane domain.

Potential N-linked glycosylation sites were found at positions 147, 155 and 237. When the protein was translated in the presence of rough endoplasmic reticulum, an increase in molecular weight was observed, consistent with glycosylation.

Further objects, features, and advantages of the present invention will readily occur to the skilled artisan provided with the disclosure above. The complete contents of all references cited in this disclosure are expressly incorporated herein by reference.

1. An isolated nucleic acid molecule comprising a polynucleotide selected from the group consisting of:
(a) a polynucleotide encoding amino acids from about 1 to about 325 of SEQ ID NO:2; about 1 to about 435 of SEQ ID NO:4; about 1 to about 339 of SEQ ID NO:6; about 1 to about 399 of SEQ ID NO:8; about 1 to about 709 of SEQ ID NO:10; about 1 to about 240 of SEQ ID NO:12; about 1 to about 613 of SEQ ID NO:14; about 1 to about 285 of SEQ ID NO:16; about 1 to about 483 of SEQ ID NO:18; about 1 to about 289 of SEQ ID NO:20; about 1 to about 585 of SEQ ID NO:22; about 1 to about 280 of SEQ ID NO:24; about 1 to about 286 of SEQ ID NO:26; about 1 to about 340 of SEQ ID NO:28; and about 1 to about 347 of SEQ ID NO:30;
(b) a polynucleotide encoding amino acids from about 2 to about 325 of SEQ ID NO:2; about 2 to about 435 of SEQ ID NO:4; about 2 to about 339 of SEQ ID NO:6; about 2 to about 399 of SEQ ID NO:8; about 2 to about 709 of SEQ ID NO:10; about 2 to about 240 of SEQ ID NO:12; about 2 to about 613 of SEQ ID NO:14; about 2 to about 285 of SEQ ID NO:16; about 2 to about 483 of SEQ ID NO:18; about 2 to about 289 of SEQ ID NO:20; about 2 to about 585 of SEQ ID NO:22; about 2 to about 280 of SEQ ID NO:24; about 2 to about 286 of SEQ ID NO:26; about 2 to about 340 of SEQ ID NO:28; and about 2 to about 347 of SEQ ID NO:30;
(c) a polynucleotide encoding amino acids from about 26 to about 273 of SEQ ID NO:2; about 25 to about 435 of SEQ ID NO:4; about 26 to about 339 of SEQ ID NO:6; about 20 to about 399 of SEQ ID NO:8; about 20 to about 240 of SEQ ID NO:12; about 24 to about 613 of SEQ ID NO:14; about 25 to about 285 of SEQ ID NO:16; about 21 to about 483 of SEQ ID NO:18; about 23 to about 289 of SEQ ID NO:20; about 14 to about 585 of SEQ ID NO:22; about 21 to about 280 of SEQ ID NO:24; about 27 to about 286 of SEQ ID NO:26; about 19 to about 340 of SEQ ID NO:28; and about 55 to about 347 of SEQ ID NO:30;
(d) the polynucleotide complement of the polynucleotide of (a), (b), or (c); and
(e) a polynucleotide at least 90% identical to the polynucleotide of (a), (b), (c), or (d).

2. An isolated nucleic acid molecule comprising at least 690 contiguous nucleotides from the coding region of any one of SEQ ID NO:1, 3, 5, 7, 9, 11, 13, 15, 17, 19, 21, 23, 25, 27 and 29.

3. The isolated nucleic acid molecule of claim 2, which comprises at least 900 contiguous nucleotides from the coding region of any one of SEQ ID NO:1, 3, 5, 7, 9, 13, 17, 21, 27, and 29.

4. The isolated nucleic acid molecule of claim 3, which comprises at least 1200 contiguous nucleotides from the coding region of any one of SEQ ID NO:3, 9, 13, 17, and 21.

5. An isolated nucleic acid molecule comprising a polynucleotide encoding a polypeptide wherein, except for at least one conservative amino acid substitution, said polypeptide has an amino acid sequence selected from the group consisting of:
(a) amino acids from about 1 to about 325 of SEQ ID NO:2; about 1 to about 435 of SEQ ID NO:4; about 1 to about 339 of SEQ ID NO:6; about 1 to about 399 of SEQ ID NO:8; about 1 to about 709 of SEQ ID NO:10; about 1 to about 240 of SEQ ID NO:12; about 1 to about 613 of SEQ ID NO:14; about 1 to about 285 of SEQ ID NO:16; about 1 to about 483 of SEQ ID NO:18; about 1 to about 289 of SEQ ID NO:20; about 1 to about 585 of SEQ ID NO:22; about 1 to about 280 of SEQ ID NO:24; about 1 to about 286 of SEQ ID NO:26; about 1 to about 340 of SEQ ID NO:28; and about 1 to about 347 of SEQ ID NO:30;
(b) amino acids from about 2 to about 325 of SEQ ID NO:2; about 2 to about 435 of SEQ ID NO:4; about 2 to about 339 of SEQ ID NO:6; about 2 to about 399 of SEQ ID NO:8; about 2 to about 709 of SEQ ID NO:10; about 2 to about 240 of SEQ ID NO:12; about 2 to about 613 of SEQ ID NO:14; about 2 to about 285 of SEQ ID NO:16; about 2 to about 483 of SEQ ID NO:18; about 2 to about 289 of SEQ ID NO:20; about 2 to about 585 of SEQ ID NO:22; about 2 to about 280 of SEQ ID NO:24; about 2 to about 286 of SEQ ID NO:26; about 2 to about 340 of SEQ ID NO:28; and about 2 to about 347 of SEQ ID NO:30; and
(c) amino acids from about 26 to about 273 of SEQ ID NO:2; about 25 to about 435 of SEQ ID NO:4; about 26 to about 339 of SEQ ID NO:6; about 20 to about 399 of SEQ ID NO:8; about 20 to about 240 of SEQ ID NO:12; about 24 to about 613 of SEQ ID NO:14; about 25 to about 285 of SEQ ID NO:16; about 21 to about 483 of SEQ ID NO:18; about 23 to about 289 of SEQ ID NO:20; about 14 to about 585 of SEQ ID NO:22; about 21 to about 280 of SEQ ID NO:24; about 27 to about 286 of SEQ ID NO:26; about 19 to about 340 of SEQ ID NO:28; and about 55 to about 347 of SEQ ID NO:30.

6. The isolated nucleic acid molecule of claim 1, which is DNA.

7. A method of making a recombinant vector comprising inserting a nucleic acid molecule of claim 1 into a vector in operable linkage to a promoter.

8. A recombinant vector produced by the method of claim 7.

9. A method of making a recombinant host cell comprising introducing the recombinant vector of claim 8 into a host cell.

10. A recombinant host cell produced by the method of claim 9.

11. A recombinant method of producing a polypeptide, comprising culturing the recombinant host cell of claim 10 under conditions such that said polypeptide is expressed and recovering said polypeptide.

12. An isolated polypeptide comprising amino acids at least 95% identical to amino acids selected from the group consisting of:
(a) amino acids from about 1 to about 325 of SEQ ID NO:2; about 1 to about 435 of SEQ ID NO:4; about 1 to about 339 of SEQ ID NO:6; about 1 to about 399 of SEQ ID NO:8; about 1 to about 709 of SEQ ID NO:10; about 1 to about 240 of SEQ ID NO:12; about 1 to about 613 of SEQ ID NO:14; about 1 to about 285 of SEQ ID NO:16; about 1 to about 483 of SEQ ID NO:18; about 1 to about 289 of SEQ ID NO:20; about 1 to about 585 of SEQ ID NO:22; about 1 to about 280 of SEQ ID NO:24; about 1 to about 286 of SEQ ID NO:26; about 1 to about 340 of SEQ ID NO:28; and about 1 to about 347 of SEQ ID NO:30;
(b) amino acids from about 2 to about 325 of SEQ ID NO:2; about 2 to about 435 of SEQ ID NO:4; about 2 to about 339 of SEQ ID NO:6; about 2 to about 399 of SEQ ID NO:8; about 2 to about 709 of SEQ ID NO:10; about 2 to about 240 of SEQ ID NO:12; about 2 to about 613 of SEQ ID NO:14; about 2 to about 285 of SEQ ID NO:16; about 2 to about 483 of SEQ ID NO:18; about 2 to about 289 of SEQ ID NO:20; about 2 to about 585 of SEQ ID NO:22; about 2 to about 280 of SEQ ID NO:24; about 2 to about 286 of SEQ ID NO:26; about 2 to about 340 of SEQ ID NO:28; and about 2 to about 347 of SEQ ID NO:30; and
(c) amino acids from about 26 to about 273 of SEQ ID NO:2; about 25 to about 435 of SEQ ID NO:4; about 26 to about 339 of SEQ ID NO:6; about 20 to about 399 of SEQ ID NO:8; about 20 to about 240 of SEQ ID NO:12; about 24 to about 613 of SEQ ID NO:14; about 25 to about 285 of SEQ ID NO:16; about 21 to about 483 of SEQ ID NO:18; about 23 to about 289 of SEQ ID NO:20; about 14 to about 585 of SEQ ID NO:22; about 21 to about 280 of SEQ ID NO:24; about 27 to about 286 of SEQ ID NO:26; about 19 to about 340 of SEQ ID NO:28; and about 55 to about 347 of SEQ ID NO:30.

13. An isolated polypeptide wherein, except for at least one conservative amino acid substitution, said polypeptide has an amino acid sequence selected from the group consisting of:
(a) amino acids from about 1 to about 325 of SEQ ID NO:2; about 1 to about 435 of SEQ ID NO:4; about 1 to about 339 of SEQ ID NO:6; about 1 to about 399 of SEQ ID NO:8; about 1 to about 709 of SEQ ID NO:10; about 1 to about 240 of SEQ ID NO:12; about 1 to about 613 of SEQ ID NO:14; about 1 to about 285 of SEQ ID NO:16; about 1 to about 483 of SEQ ID NO:18; about 1 to about 289 of SEQ ID NO:20; about 1 to about 585 of SEQ ID NO:22; about 1 to about 280 of SEQ ID NO:24; about 1 to about 286 of SEQ ID NO:26; about 1 to about 340 of SEQ ID NO:28; and about 1 to about 347 of SEQ ID NO:30;
(b) amino acids from about 2 to about 325 of SEQ ID NO:2; about 2 to about 435 of SEQ ID NO:4; about 2 to about 339 of SEQ ID NO:6; about 2 to about 399 of SEQ ID NO:8; about 2 to about 709 of SEQ ID NO:10; about 2 to about 240 of SEQ ID NO:12; about 2 to about 613 of SEQ ID NO:14; about 2 to about 285 of SEQ ID NO:16; about 2 to about 483 of SEQ ID NO:18; about 2 to about 289 of SEQ ID NO:20; about 2 to about 585 of SEQ ID NO:22; about 2 to about 280 of SEQ ID NO:24; about 2 to about 286 of SEQ ID NO:26; about 2 to about 340 of SEQ ID NO:28; and about 2 to about 347 of SEQ ID NO:30; and
(c) amino acids from about 26 to about 273 of SEQ ID NO:2; about 25 to about 435 of SEQ ID NO:4; about 26 to about 339 of SEQ ID NO:6; about 20 to about 399 of SEQ ID NO:8; about 20 to about 240 of SEQ ID NO:12; about 24 to about 613 of SEQ ID NO:14; about 25 to about 285 of SEQ ID NO:16; about 21 to about 483 of SEQ ID NO:18; about 23 to about 289 of SEQ ID NO:20; about 14 to about 585 of SEQ ID NO:22; about 21 to about 280 of SEQ ID NO:24; about 27 to about 286 of SEQ ID NO:26; about 19 to about 340 of SEQ ID NO:28; and about 55 to about 347 of SEQ ID NO:30.

14. An isolated polypeptide comprising amino acids selected from the group consisting of:
(a) amino acids from about 1 to about 325 of SEQ ID NO:2; about 1 to about 435 of SEQ ID NO:4; about 1 to about 339 of SEQ ID NO:6; about 1 to about 399 of SEQ ID NO:8; about 1 to about 709 of SEQ ID NO:10; about 1 to about 240 of SEQ ID NO:12; about 1 to about 613 of SEQ ID NO:14; about 1 to about 285 of SEQ ID NO:16; about 1 to about 483 of SEQ ID NO:18; about 1 to about 289 of SEQ ID NO:20; about 1 to about 585 of SEQ ID NO:22; about 1 to about 280 of SEQ ID NO:24; about 1 to about 286 of SEQ ID NO:26; about 1 to about 340 of SEQ ID NO:28; and about 1 to about 347 of SEQ ID NO:30;
(b) amino acids from about 2 to about 325 of SEQ ID NO:2; about 2 to about 435 of SEQ ID NO:4; about 2 to about 339 of SEQ ID NO:6; about 2 to about 399 of SEQ ID NO:8; about 2 to about 709 of SEQ ID NO:10; about 2 to about 240 of SEQ ID NO:12; about 2 to about 613 of SEQ ID NO:14; about 2 to about 285 of SEQ ID NO:16; about 2 to about 483 of SEQ ID NO:18; about 2 to about 289 of SEQ ID NO:20; about 2 to about 585 of SEQ ID NO:22; about 2 to about 280 of SEQ ID NO:24; about 2 to about 286 of SEQ ID NO:26; about 2 to about 340 of SEQ ID NO:28; and about 2 to about 347 of SEQ ID NO:30; and
(c) amino acids from about 26 to about 273 of SEQ ID NO:2; about 25 to about 435 of SEQ ID NO:4; about 26 to about 339 of SEQ ID NO:6; about 20 to about 399 of SEQ ID NO:8; about 20 to about 240 of SEQ ID NO:12; about 24 to about 613 of SEQ ID NO:14; about 25 to about 285 of SEQ ID NO:16; about 21 to about 483 of SEQ ID NO:18; about 23 to about 289 of SEQ ID NO:20; about 14 to about 585 of SEQ ID NO:22; about 21 to about 280 of SEQ ID NO:24; about 27 to about 286 of SEQ ID NO:26; about 19 to about 340 of SEQ ID NO:28; and about 55 to about 347 of SEQ ID NO:30.

15. An epitope-bearing portion of the polypeptide of any one of SEQ ID NO:2, 4, 6, 8, 10, 12, 14, 16, 18, 20, 22, 24, 26, 28 and 30.

16. The epitope-bearing portion of claim 15, which comprises about 8 to 25 contiguous amino acids of any one of SEQ ID NO:2, 4, 6, 8, 10, 12, 14, 16, 18, 20, 22, 24, 26, 28 and 30.

17. The epitope-bearing portion of claim 15, which comprises about 10 to 15 contiguous amino acids of any one of SEQ ID NO:2, 4, 6, 8, 10, 12, 14, 16, 18, 20, 22, 24, 26, 28, and 30.

18. An isolated antibody that binds specifically to the polypeptide of claim 12.

19. An isolated antibody that binds specifically to a polypeptide of claim 13.

20. An isolated antibody that binds specifically to the polypeptide of claim 14.

21. An isolated nucleic acid molecule comprising a polynucleotide selected from the group consisting of:
(a) a polynucleotide encoding amino acids from about 1 to about 13 and about 34 to about 585 of SEQ ID NO:22, wherein said amino acids about 13 and about 34 are joined by a peptide bond;
(b) a polynucleotide encoding amino acids from about 1 to about 20 and about 180 to about 280 of SEQ ID NO:24, wherein said amino acids about 20 and about 180 are joined by a peptide bond;
(c) a polynucleotide encoding amino acids from about 1 to about 179 of SEQ ID NO:24;
(d) a polynucleotide encoding amino acids from about 21 to about 206 of SEQ ID NO:24;
(e) a polynucleotide encoding amino acids from about 1 to about 26 and about 61 to about 286 of SEQ ID NO:26, wherein said amino acids about 26 and about 61 are joined by a peptide bond;
(f) a polynucleotide encoding amino acids from about 27 to about 53 and about 257 to about 286 of SEQ ID NO:26, wherein said amino acids about 27 and about 257 are joined by a peptide bond;
(g) a polynucleotide encoding amino acids from about 1 to about 18 and about 144 to about 340 of SEQ ID NO:28, wherein said amino acids about 18 and about 144 are joined by a peptide bond;

22. A fusion protein comprising a first protein segment and a second protein segment fused together by means of a peptide bond, wherein the first protein segment consists of at least 8 contiguous amino acids of an amino acid sequence selected from the group consisting of the amino acid sequences shown in SEQ ID NOS:2, 4, 6, 8, 10, 12, 14, 16, 18, 20, 22, 24, 26, 28 and 30.

23. The fusion protein of claim 22 wherein the first protein segment consists of an amino acid sequence selected from the group consisting of amino acid sequence shown in SEQ ID NOS: 2, 4, 6, 8, 10, 12, 14, 16, 18, 20, 22, 24, 26, 28 and 30.


SEQUENCE LISTING (30 sequences)

SEQ ID NO: 1 - DNA, 1313 bp, Homo sapiens
  CDS (163)...(1137)
gaattcggcacgaggatttccacccagaagacagagaaggagccagtggtcatggaatgggctggggtcaaagactgggtgcctgggagctgaggcagccaccgtttcagcctggccagccctctggaccccgaggttggaccctactgtgacacacctaccatgcggacactcttcaacctcctctggcttgccctggcctgcagccctgttcacactaccctgtcaaagtcagatgccaaaaaagccgcctcaaagacgctgctggagaagagtcagttttcagataagccggtgcaagaccggggtttggtggtgacggacctcaaagctgagagtgtggttcttgagcatcgcagctactgctcggcaaaggcccgggacagacactttgctggggatgtactgggctatgtcactccatggaacagccatggctacgatgtcaccaaggtctttgggagcaagttcacacagatctcacccgtctggctgcagctgaagagacgtggccgtgagatgtttgaggtcacgggcctccacgacgtggaccaagggtggatgcgagctgtcaggaagcatgccaagggcctgcacatagtgcctcggctcctgtttgaggactggacttacgatgatttccggaacgtcttagacagtgaggatgagatagaggagctgagcaagaccgtggtccaggtggcaaagaaccagcatttcgatggcttcgtggtggaggtctggaaccagctgctaagccagaagcgcgtgggcctcatccacatgctcacccacttggccgaggctctgcaccaggcccggctgctggccctcctggtcatcccgcctgccatcacccccgggaccgaccagctgggcatgttcacgcacaaggagtttgagcagctggcccccgtgctggatggtttcagcctcatgacctacgactactctacagcgcatcagcctggccctaatgcacccctgtcctgggttcgagcctgcgtccaggtcctggacccgaagtccaagtggcgaagcaaaatcctcctggggctcaacttctatggtatggactacgcgacctccaaggatgcccgtgagcctgttgtcggggccagtgttcatggagcctcttgagtctgcattcgtactcttagcaaacttggaaaatttgaggccaaattcttcaaatattttttctgtttcttctcttctgtcttctcgtttggagaccacaaacactagatccattgaatttgtcccacagctcacgaatacaccttttaccttttaaaaaaaaaaaaaaaaaa

SEQ ID NO: 2 - protein, 325 aa, Homo sapiens
MRTLFNLLWLALACSPVHTTLSKSDAKKAASKTLLEKSQFSDKPVQDRGLVVTDLKAESVVLEHRSYCSAKARDRHFAGDVLGYVTPWNSHGYDVTKVFGSKFTQISPVWLQLKRRGREMFEVTGLHDVDQGWMRAVRKHAKGLHIVPRLLFEDWTYDDFRNVLDSEDEIEELSKTVVQVAKNQHFDGFVVEVWNQLLSQKRVGLIHMLTHLAEALHQARLLALLVIPPAITPGTDQLGMFTHKEFEQLAPVLDGFSLMTYDYSTAHQPGPNAPLSWVRACVQVLDPKSKWRSKILLGLNFYGMDYATSKDAREPVVGASVHGAS

SEQ ID NO: 3 - DNA, 1941 bp, Homo sapiens
  CDS (262)...(1566)
gaattcggcacgaggccgcctgggccccgccgagcggagctagcgccgcgcgcagagcacacgctcgcgctccagctcccctcctgcgcggttcatgactgtgtcccctgaccgcagcctctgcgagcccccgccgcaggaccacggcccgctccccgccgccgcgagggccccgagcgaaggaaggaagggaggcgcgctgtgcgccccgcggagcccgcgaaccccgctcgctgccggctgcccagcctggctggcaccatgctgcccgcgcgctgcgcccgcctgctcacgccccacttgctgctggtgttggtgcagctgtcccctgctcgcggccaccgcaccacaggccccaggtttctaataagtgaccgtgacccacagtgcaacctccactgctccaggactcaacccaaacccatctgtgcctctgatggcaggtcctacgagtccatgtgtgagtaccagcgagccaagtgccgagacccgaccctgggcgtggtgcatcgaggtagatgcaaagatgctggccagagcaagtgtcgcctggagcgggctcaagccctggagcaagccaagaagcctcaggaagctgtgtttgtcccagagtgtggcgaggatggctcctttacccaggtgcagtgccatacttacactgggtactgctggtgtgtcaccccggatgggaagcccatcagtggctcttctgtgcagaataaaactcctgtatgttcaggttcagtcaccgacaagcccttgagccagggtaactcaggaaggaaagatgacgggtctaagccgacacccacgatggagacccagccggtgttcgatggagatgaaatcacagccccaactctatggattaaacacttggtgatcaaggactccaaactgaacaacaccaacataagaaattcagagaaagtctattcgtgtgaccaggagaggcagagtgccctggaagaggcccagcagaatccccgtgagggtattgtcatccctgaatgtgcccctgggggactctataagccagtgcaatgccaccagtccactggctactgctggtgtgtgctggtggacacagggcgcccgctgcctgggacctccacacgctacgtgatgcccagttgtgagagcgacgccagggccaagactacagaggcggatgaccccttcaaggacagggagctaccaggctgtccagaagggaagaaaatggagtttatcaccagcctactggatgctctcaccactgacatggttcaggccattaactcagcagcgcccactggaggtgggaggttctcagagccagaccccagccacaccctggaggagcgggtagtgcactggtatttcagccagctggacagcaatagcagcaacgacattaacaagcgggagatgaagcccttcaagcgctacgtgaagaagaaagccaagcccaaggaatgtgcccggcgtttcaccgactactgtgacctgaacaaagacaaggtcatttcactgcctgagctgaagggctgcctgggtgttagcaaagaagtaggacgcctcgtctaaggagcagaaaacccaagggcaggtggagagtccagggaggcaggatggatcaccagacacctaaccttcagcgttgcccatggccctgccacatcccgtgtaacataagtggtgcccaccatgtttgcacttttaataactcttacttgcgtgttttgtttttggtttcattttaaaacaccaatatctaataccacagtgggaaaaggaaagggaagaaagactttattctctctcttattgtaagtttttggatctgctactgacaacttttagagggttttgggggggtgggggagggtgttgttggggctgagaagaaagagatttatatgctgtatataaatatatatgtaaaaaaaaaaaaaaaaa

SEQ ID NO: 4 - protein, 435 aa, Homo sapiens
MLPARCARLLTPHLLLVLVQLSPARGHRTTGPRFLISDRDPQCNLHCSRTQPKPICASDGRSYESMCEYQRAKCRDPTLGVVHRGRCKDAGQSKCRLERAQALEQAKKPQEAVFVPECGEDGSFTQVQCHTYTGYCWCVTPDGKPISGSSVQNKTPVCSGSVTDKPLSQGNSGRKDDGSKPTPTMETQPVFDGDEITAPTLWIKHLVIKDSKLNNTNIRNSEKVYSCDQERQSALEEAQQNPREGIVIPECAPGGLYKPVQCHQSTGYCWCVLVDTGRPLPGTSTRYVMPSCESDARAKTTEADDPFKDRELPGCPEGKKMEFITSLLDALTTDMVQAINSAAPTGGGRFSEPDPSHTLEERVVHWYFSQLDSNSSNDINKREMKPFKRYVKKKAKPKECARRFTDYCDLNKDKVISLPELKGCLGVSKEVGRLV

SEQ ID NO: 5 - DNA, 1839 bp, Homo sapiens
  CDS (40)...(1056)
gaattcggcacgagcccggcgccatcttcatcgagcgccatggccgcagcctgcgggccgggagcggccgggtactgcttgctcctcggcttgcatttgtttctgctgaccgcgggccctgccctgggctggaacgaccctgacagaatgttgctgcgggatgtaaaagctcttaccctccactatgaccgctataccacctcccgcaggctggatcccatcccacagttgaaatgtgttggaggcacagctggttgtgattcttataccccaaaagtcatacagtgtcagaacaaaggctgggatgggtatgatgtacagtgggaatgtaagacggacttagatattgcatacaaatttggaaaaactgtggtgagctgtgaaggctatgagtcctctgaagaccagtatgtactaagaggttcttgtggcttggagtataatttagattatacagaacttggcctgcagaaactgaaggagtctggaaagcagcacggctttgcctctttctctgattattattataagtggtcctcggcggattcctgtaacatgagtggattgattaccatcgtggtactccttgggatcgcctttgtagtctataagctgttcctgagtgacgggcagtattctcctccaccgtactctgagtatcctccattttcccaccgttaccagagattcaccaactcagcaggacctcctcccccaggctttaagtctgagttcacaggaccacagaatactggccatggtgcaacttctggttttggcagtgcttttacaggacaacaaggatatgaaaattcaggaccagggttctggacaggcttgggaactggtggaatactaggatatttgtttggcagcaatagagcggcaacacccttctcagactcgtggtactacccgtcctatcctccctcctaccctggcacgtggaatagggcttactcaccccttcatggaggctcgggcagctattcggtatgttcaaactcagacacgaaaaccagaactgcatcaggatatggtggtaccaggagacgataaagtagaaagttggagtcaaacactggatgcagaaattttggatttttcatcactttctctttagaaaaaaagtactacctgttaacaattgggaaaaggggatattcaaaagttctgtggtgttatgtccagtgtagctttttgtattctattatttgaggctaaaagttgatgtgtgacaaaatacttatgtgttgtatgtcagtgtaacatgcagatgtatattgcagtttttgaaagtgatcattactgtggaatgctaaaaatacattaatttctaaaacctgtgatgccctaagaagcattaagaatgaaggtgttgtactaatagaaactaagtacagaaaatttcagttttaggtggttgtagctgatgagttattacctcatagagactataatattctatttggtattatattatttgatgtttgctgttcttcaaacatttaaatcaagctttggactaattatgctaatttgtgagttctgatcacttttgagctctgaagctttgaatcattcagtggtggagatggccttctggtaactgaatattaccttctgtaggaaaaggtggaaaataagcatctagaaggttgttgtgaatgactctgtgctggcaaaaatgcttgaaacctctatatttctttcgttcataagaggtaaaggtcaaatttttcaacaaaagtcttttaataacaaaagcatgcagttctctgtgaaatctcaaatattgttgtaatagtctgtttcaatcttaaaaaaaaaaaaaaaaaa

SEQ ID NO: 6 - protein, 339 aa, Homo sapiens
MAAACGPGAAGYCLLLGLHLFLLTAGPALGWNDPDRMLLRDVKALTLHYDRYTTSRRLDPIPQLKCVGGTAGCDSYTPKVIQCQNKGWDGYDVQWECKTDLDIAYKFGKTVVSCEGYESSEDQYVLRGSCGLEYNLDYTELGLQKLKESGKQHGFASFSDYYYKWSSADSCNMSGLITIVVLLGIAFVVYKLFLSDGQYSPPPYSEYPPFSHRYQRFTNSAGPPPPGFKSEFTGPQNTGHGATSGFGSAFTGQQGYENSGPGFWTGLGTGGILGYLFGSNRAATPFSDSWYYPSYPPSYPGTWNRAYSPLHGGSGSYSVCSNSDTKTRTASGYGGTRRR

SEQ ID NO: 7 - DNA, 1831 bp, Homo sapiens
  CDS (90)...(1286)
gaattcggcacgagctggcggccaccagaagtttgagcctctttggtagcaggaggctggaagaaaggacagaagtagctctggctgtgatggggatcttactgggcctgctactcctggggcacctaacagtggacacttatggccgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaactctctgtctccaagcccacagtgacaactggcagcggttatggcttcacggtgccccagggaatgaggattagccttcaatgccaggctcggggttctcctcccatcagttatatttggtataagcaacagactaataaccaggaacccatcaaagtagcaaccctaagtaccttactcttcaagcctgcggtgatagccgactcaggctcctatttctgcactgccaagggccaggttggctctgagcagcacagcgacattgtgaagtttgtggtcaaagactcctcaaagctactcaagaccaagactgaggcacctacaaccatgacataccccttgaaagcaacatctacagtgaagcagtcctgggactggaccactgacatggatggctaccttggagagaccagtgctgggccaggaaagagcctgcctgtctttgccatcatcctcatcatctccttgtgctgtatggtggtttttaccatggcctatatcatgctctgtcggaagacatcccaacaagagcatgtctacgaagcagccagggcacatgccagagaggccaacgactctggagaaaccatgagggtggccatcttcgcaagtggctgctccagtgatgagccaacttcccagaatctgggcaacaactactctgatgagccctgcataggacaggagtaccagatcatcgcccagatcaatggcaactacgcccgcctgctggacacagttcctctggattatgagtttctggccactgagggcaaaagtgtctgttaaaaatgccccattaggccaggatctgctgacataattgcctagtcagtccttgccttctgcatggccttcttccctgctacctctcttcctggatagcccaaagtgtccgcctaccaacactggagccgctgggagtcactggctttgccctggaatttgccagatgcatctcaagtaagccagctgctggatttggctctgggcccttctagtatctctgccgggggcttctggtactcctctctaaataccagagggaagatgcccatagcactaggacttggtcatcatgcctacagacactattcaactttggcatcttgccaccagaagacccgagggaggctcagctctgccagctcagaggaccagctatattcaggatcatttctctttcttcagggccagacagcttttaattgaaattgttatttcacaggccagggttcagttctgctcctccactataagtctaatgttctgactctctcctggtgctcaataaatatctaatcataacagcaaaaaaaaaaaaaaaaaaa

SEQ ID NO: 8 - protein, 399 aa, Homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 9 - DNA, 4222 bp, Homo sapiens
  CDS (238)...(2364)
ggatccaaagaattcggcacgagagactccctaacctgtgtctggacaagtctgatgtcctgtgtggcccaagaagaactgaccccgtgtctggagctcccaccgttattgcatccctgctgtggctcacctgctgctgtctccaggagcccctgagaagatttgcctcctctcccctgctaagctccaggtcctgagattgaattaggggctggagctcactgcactccagcagtcatgggacccaggatagggccagcgggtgaggtaccccaggtaccagacaaggaaaccaaagccacaatgggcacagaaaacacacctggaggcaaagccagcccagaccctcaggacgtgcggccaagtgtgttccataacatcaagctgttcgttctgtgccacagcctgctgcagctggcgcagctcatgatctccggctacctaaagagctccatctccacagtggagaagcgcttcggcctctccagccagacgtcggggctgctggcctccttcaacgaggtggggaacacagccttgattgtgtttgtgagctattttggcagccgggtgcaccgaccccgaatgattggctatggggctatccttgtggccctggcgggcctgctcatgactctcccgcacttcatctcggagccataccgctacgacaacaccagccctgaggatatgccacaggacttcaaggcttccctgtgcctgcccacaacctcggccccagcctcggccccctccaatggcaactgctcaagctacacagaaacccagcatctgagtgtggtggggatcatgttcgtggcacagaccctgctgggcgtgggcggggtgcccattcagccctttggcatctcctacatcgatgactttgcccacaacagcaactcgcccctctacctcgggatcctgtttgcagtgaccatgatggggccaggcctggcctttgggctgggcagcctcatgctgcgcctttatgtggacattaaccagatgccagaaggtggtatcagcctgaccataaaggacccccgatgggtgggtgcctggtggctgggtttcctcatcgctgccggtgcagtggccctggctgccatcccctacttcttcttccccaaggaaatgcccaaggaaaaacgtgagcttcagtttcggcgaaaggtcttagcagtcacagactcacctgccaggaagggcaaggactctccctctaagcagagccctggggagtccacgaagaagcaggatggcctagtccagattgcaccaaacctgactgtgatccagttcattaaagtcttccccagggtgctgctgcagaccctacgccaccccatcttcctgctggtggtcctgtcccaggtatgcttgtcatccatggctgcgggcatggccaccttcctgcccaagttcctggagcgccagttttccatcacagcctcctacgccaacctgctcatcggctgcctctccttcccttcggtcatcgtgggcatcgtggtgggtggcgtcctggtcaagcggctccacctgggccctgtgggatgcggtgccctttgcctgctggggatgctgctgtgcctcttcttcagcctgccgctcttctttatcggctgctccagccaccagattgcgggcatcacacaccagaccagtgcccaccctgggctggagctgtctccaagctgcatggaggcctgctcctgcccattggacggctttaaccctgtctgcgaccccagcactcgtgtggaatacatcacaccctgccacgcaggctgctcaagctgggtggtccaggatgctctggacaacagccaggttttctacaccaactgcagctgcgtggtggagggcaaccccgtgctggcaggatcctgcgactcaacgtgcagccatctggtggtgcccttcctgctcctggtcagcctgggctcggccctggcctgtctcacccacacaccctccttcatgctcatcctaagaggagtgaagaaagaagacaagactttggctgtgggcatccagttcatgttcctgaggattttggcctggatgcccagccccgtgatccacggcagcgccatcgacaccacctgtgtgcactgggccctgagctgtgggcgtcgagctgtctgtcgctactacaataatgacctgctccgaaaccggttcatcggcctccagttcttcttcaaaacaggttctgtgatctgcttcgccttagttttggctgtcctgaggcagcaggacaaagaggcaaggaccaaagagagcagatccagccctgccgtagagcagcaattgctagtgtcggggccagggaagaagccagaggattcccgagtgtgagctgtcttggggccccacctggccaagagtagcagccacagcagtacctcctctgagtcctttgcccaagattgggtgtcaagagccctgtgttccattctggctcctccactaaattgctgtgtgacttcaggcaagacattgatcctctctcagcctttgcttgctagtctgaaccaaagagttgtttgggcatttgctgtgttggccatttctggagcaagagggtcttcttcctccttcccccagccagccagctgtcctggggccaggctttcctgggtggaaagaagtatacctttccctggggccctaggatagcaaagtgagccatagtgggccaggctgccctccatgctgggccccagcccaggtctgcactcgcctggatcaccttctttgagccttagccatctcctgtcaggtaggaatgaacttgccagccttcaggctcgttcagctatgaccatctgtgcggtcagggtacactcagctctcctccccaactccagcagcctttaagaagtgtccctttggcgccccctggaggcagagcactgagctggaccctgggtagactcccacagggaggacggagctggcctcaggagtgggacacccagacttggcagggccttcaagaggcctgtgtgggggccccaggaatccttagctgaagcggggagactcactctccatctcaggaaattctagcccttgccctcagggagccacggttgagggtgaggcccaacacctgccttagggccctgggtgggcaagtctgggccctggggtagggagggagactcaggcccacacttgggtattttctaatttcagacaaacacacactcagcgcgcactcactgattcctacacattgccaagatttcacacatgtgaccaggggccaccaaagtccctgtgacctttgtgactaggatcctaatttctctattttctcctgggtgcctgggtctgtgtcacctggggcagtgtggataatgtttagttctgtgacactgttttttgggggtggcacctggttctccgatgcctgggctggtgtcaggcccaggactgtagtgctgggagcagtaaagctcagctctgtgtaatgagtgatgctatggcttgctcgtgtcttatgatccaatccttttctacatcagcccttgttttgttttatggctagtcttatctggcctggttatttccttgcggggaggagagggtttgctaatctgctcccagcccaacctattaccaccccacctcgctgggacctactgctcgggaggcagcagacagggagccaccagcagtggcttcctggccctgtgctgggggtggggggaagctgggggcacatgtggcccttgccttctgagcagctcccagtgccagggctttgagactttcccacatgataaaagaaaagggaggtacagaagttccaattccctttttattttgctggttggtatctgtaaatgtttaataaatatctgagcatgtatctatcaacgccaagaatttcaaagtctccttcaacaatatgaggcttttaggatgtttatattccttcatccctcttgtttcccaggttttgcagggaaaaaaagtctggaattatagatacagcttattattaaatttgttcttgcataatgtctcttctattacaaaaattctttcttcataaactgcattagaggtttgcaacaaccacatcatttccattaacttagatttaggttttactggattcattgctcaccattattgcttgtatattacatcttttccaatctttaaaaaaaaaaaaaaaaaactcgagagtacttctaga

SEQ ID NO: 10 - protein, 709 aa, Homo sapiens
MGPRIGPAGEVPQVPDKETKATMGTENTPGGKASPDPQDVRPSVFHNIKLFVLCHSLLQLAQLMISGYLKSSISTVEKRFGLSSQTSGLLASFNEVGNTALIVFVSYFGSRVHRPRMIGYGAILVALAGLLMTLPHFISEPYRYDNTSPEDMPQDFKASLCLPTTSAPASAPSNGNCSSYTETQHLSVVGIMFVAQTLLGVGGVPIQPFGISYIDDFAHNSNSPLYLGILFAVTMMGPGLAFGLGSLMLRLYVDINQMPEGGISLTIKDPRWVGAWWLGFLIAAGAVALAAIPYFFFPKEMPKEKRELQFRRKVLAVTDSPARKGKDSPSKQSPGESTKKQDGLVQIAPNLTVIQFIKVFPRVLLQTLRHPIFLLVVLSQVCLSSMAAGMATFLPKFLERQFSITASYANLLIGCLSFPSVIVGIVVGGVLVKRLHLGPVGCGALCLLGMLLCLFFSLPLFFIGCSSHQIAGITHQTSAHPGLELSPSCMEACSCPLDGFNPVCDPSTRVEYITPCHAGCSSWVVQDALDNSQVFYTNCSCVVEGNPVLAGSCDSTCSHLVVPFLLLVSLGSALACLTHTPSFMLILRGVKKEDKTLAVGIQFMFLRILAWMPSPVIHGSAIDTTCVHWALSCGRRAVCRYYNNDLLRNRFIGLQFFFKTGSVICFALVLAVLRQQDKEARTKESRSSPAVEQQLLVSGPGKKPEDSRV

SEQ ID NO: 11 - DNA, 960 bp, Homo sapiens
  CDS (78)...(797)
gaattcggcacgagggccggcctccgcccggccccgagggcaggctctccccggaggctcagccccctctgctccccatgggcaactgccaggcagggcacaacctgcacctgtgtctggcccaccacccacctctggtctgtgccactttgatcctgctgctccttggcctctctggcctgggccttggcagcttcctcctcacccacaggactggcctgcgcagccctgacatcccccaggactgggtctcttttttgagatcttttggccagctgaccctgtgtcccaggaatgggacagtcacagggaagtggcgagggtctcacgtcgtgggcttgctgaccaccttgaacttcggagacggtccagacaggaacaagacccggacattccaggccacagtcctgggaagtcagatgggattgaaaggatcttctgcaggacaactggtccttatcacagccagggtgaccacagaaaggactgcaggaacctgcctatattttagtgctgttccaggaatcctaccctccagccagccacccatatcctgctcagaggagggggctggaaatgccaccctgagccctagaatgggtgaggaatgtgttagtgtctggagccatgaaggccttgtgctgaccaagctgctcacctcggaggagctggctctgtgtggctccaggctgctggtcttgggctccttcctgcttctcttctgtggccttctctgctgtgtcactgctatgtgcttccacccgcgccgggagtcccactggtctagaacccggctctgagggcactggcctagttcccgacttgtttctcaggtgtgaatcaacttcttgggccttggctctgagttggaaaaggttttagaaaaagtgaagagctggaatgtgggggaaaataaaaagcttttttgcccaaaaaaaaaaaaaaaaaaaaaaaaaaaaa

SEQ ID NO: 12 - protein, 240 aa, Homo sapiens
MGNCQAGHNLHLCLAHHPPLVCATLILLLLGLSGLGLGSFLLTHRTGLRSPDIPQDWVSFLRSFGQLTLCPRNGTVTGKWRGSHVVGLLTTLNFGDGPDRNKTRTFQATVLGSQMGLKGSSAGQLVLITARVTTERTAGTCLYFSAVPGILPSSQPPISCSEEGAGNATLSPRMGEECVSVWSHEGLVLTKLLTSEELALCGSRLLVLGSFLLLFCGLLCCVTAMCFHPRRESHWSRTRL

SEQ ID NO: 13 - DNA, 2832 bp, Homo sapiens
  CDS (317)...(2155)
ggatccaaagaattcggcacgaggctggctcagcccagagtccctgtctcccgcccgccggcccgagccgccgcccctcccccgcctcccgtgcgcccgggacaatcctcgccttgtctgtggcgccggcatctggagctttctgtagcctccggatacgcctttttttcagggcgtagccccagccaagctgctccccgcggcggccgcacagcagcccgagcgccccctttccggagctcccctccggagctgggatccaggcgcgtagcggagatcccaggatcctgggtgctgtctgggcccgctccccaccatgaccttcttggggcctggaccccggttcctgctgctgctgccgctgctgctgccccctgcggcctcagcctccgaccggccccggggccgagacccggtcaacccagagaagctgctggtgatcactgtggccacagctgaaaccgaggggtacctgcgtttcctgcgctctgcggagttcttcaactacactgtgcggaccctgggcctgggagaggagtggcgagggggtgatgtggctcgaacagttggtggaggacagaaggtccggtggttaaagaaggaaatggagaaatacgctgaccgggaggatatgatcatcatgtttgtggatagctacgacgtgattctggccggcagccccacagagctgctgaagaagttcgtccagagtggcagccgcctgctcttctctgcagagagcttctgctggcccgagtgggggctggcggagcagtaccctgaggtgggcacggggaagcgcttcctcaattctggtggattcatcggttttgccaccaccatccaccaaatcgtgcgccagtggaagtacaaggatgatgacgacgaccagctgttctacacacggctctacctggacccaggactgagggagaaactcagccttaatctggatcataagtctcggatctttcagaacctcaacggggctttagatgaagtggttttaaagtttgatcggaaccgtgtgcgtatccggaacgtggcctacgacacgctccccattgtggtccatggaaacggtcccactaagctgcagctcaactacctgggaaactacgtccccaatggctggactcctgagggaggctgtggcttctgcaaccaggaccggaggacactcccgggggggcagcctcccccccgggtgtttctggccgtgtttgtggaacagcctactccgtttctgccccgcttcctgcagcggctgctactcctggactatccccccgacagggtcacccttttcctgcacaacaacgaggtcttccatgaaccccacatcgctgactcctggccgcagctccaggaccacttctcagctgtgaagctcgtggggccggaggaggctctgagcccaggcgaggccagggacatggccatggacctgtgtcggcaggaccccgagtgtgagttctacttcagcctggacgccgacgctgtcctcaccaacctgcagaccctgcgtatcctcattgaggagaacaggaaggtgatcgcccccatgctgtcccgccacggcaagctgtggtccaacttctggggcgccctgagccccgatgagtactacgcccgctccgaggactacgtggagctggtgcagcggaagcgagtgggtgtgtggaatgtaccatacatctcccaggcctatgtgatccggggtgataccctgcggatggagctgccccagagggatgtgttctcgggcagtgacacagacccggacatggccttctgtaagagctttcgagacaagggcatcttcctccatctgagcaatcagcatgaatttggccggctcctggccacttccagatacgacacggagcacctgcaccccgacctctggcagatcttcgacaaccccgtcgactggaaggagcagtacatccacgagaactacagccgggccctggaagggaaggaatcgtggagcagccatgcccggacgtgtactggttcccactgctgtcagaacaaatgtgtgatgagctggtggcagagatggagcactacggccagtggtcaggcggccggcatgaggattcaaggctggctggaggctacgagaatgtgcccaccgtggacatccacatgaagcaggtggggtacgaggaccagtggctgcagctgctgcggacgtatgtgggccccatgaccgagagcctgtttcccggttaccacaccaaggcgcgggcggtgatgaactttgtggttcgctaccggccagacgagcagccgtctctgcgggccacaccacgactcatccaccttcaccctcaacgttgccctcaaccacaagggcctggactatgagggaggtggctgccgcttcctgcgctacgactgtgtgatctcctccccgaggaagggctgggcactcctgcaccccggccgcctcacccactaccacgaggggctgccaacgacctggggcacacgctacatcatggtgtcctttgtcgacccctgacactcaaccactctgccaaacctgccctgccattgtgcctttttagggggcctggcccccgtcctgggagttgggggatgggtctctctgtctccccacttcctgagttcatgttccgcgtgcctgaactgaatatgtcaccttgctcccaagacacggccctctcaggaagctcccggagtccccgcctctctcctccgcccacaggggttcgtgggcacagggcttctggggactccccgcgtgataaattattaatgttccgcagtctcactctgaataaaggacagtttgtaagt

SEQ ID NO: 14 - protein, 613 aa, Homo sapiens
MTFLGPGPRFLLLLPLLLPPAASASDRPRGRDPVNPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPIVVHGNGPTKLQLNYLGNYVPNGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLATSRYDTEHLHPDLWQIFDNPVDWKEQYIHENYSRALEGKESWSSHARTCTGSHCCQNKCVMSWWQRWSTTASGQAAGMRIQGWLEATRMCPPWTST

SEQ ID NO: 15 - DNA, 3030 bp, Homo sapiens
  CDS (31)...(885)
ggatccaaagaattcggcacgagcgccgcgatgcccgcgcgcccaggacgcctcctcccgctgctggcccggccggcggccctgactgcgctgctgctgctgctgctgggccatggcggcggcgggcgctggggcgcccgggcccaggaggcggcggcggcggcggcggacgggccccccgcggcagacggcgaggacggacaggacccgcacagcaagcacctgtacacggccgacatgttcacgcacgggatccagagcgccgcgcacttcgtcatgttcttcgcgccctggtgtggacactgccagcggctgcagccgacttggaatgacctgggagacaaatacaacagcatggaagatgccaaagtctatgtggctaaagtggactgcacggcccactccgacgtgtgctccgcccagggggtgcgaggataccccaccttaaagcttttcaagccaggccaagaagctgtgaagtaccagggtcctcgggacttccagacactggaaaactggatgctgcagacactgaacgaggagccagtgacaccagaaccggaagtggaaccgccagtgcccccgagctcaagcaagggctgtatgagctctcagcaagcaactttgagctgcacgttgcacaaggcgaccactttatcaagttcttcgctccgtggtgtggtcactgcaaagccctggctccaacctgggagcagctggctctgggccttgaacattccgaaactgtcaagattggcaaggttgattgtacacagcactatgaactctgctccggaaaccaggttcgtggctatcccactcttctctggttccgagatgggaaaaaggtggatcagtacaagggaaagcgggatttggagtcactgagggagtacgtggagtcgcagctgcagcgcacagagactggagcgacggagaccgtcacgccctcagaggccccggtgctggcagctgagcccgaggctgacaagggcactgtgttggcactcactgaaaataacttcgatgacaccattgcagaaggaataaccttcatcaagttttatgctccatggtgtggtcattgtaagactctggctcctacttgggaggaactctctaaaaaggaattccctggtctggcgggggtcaagatcgccgaagtagactgcactgctgaacggaatatctgcagcaagtattcggtacgaggctaccccacgttattgcttttccgaggagggaagaaagtcagtgagcacagtggaggcagagaccttgactcgttacaccgctttgtcctgagccaagcgaaagacgaactttaggaacacagttggaggtcacctctcctgcccagctcccgcaccctgcgtttaggagttcagtcccacagaggccactgggttcccagtggtggctgttcagaaagcagaacatactaagcgtgaggtatcttctttgtgtgtgtgttttccaagccaacacactctacagattctttattaagttaagtttctctaagtaaatgtgtaactcatggtcactgtgtaaacattttcagtggcgatatatcccctttgaccttctcttgatgaaatttacatggtttcctttgagactaaaatagcattgagggaaatgaaattgctggactatttgtggctcctgagttgagtgattttggtgaaagaaagcacatccaaagcatagtttacctgcccacgagttctggaaaggtggccttgtggcagtattgacgttcctctgatcttaaggtcacagttgactcaatactgtgttggtccgtagcatggagcagattgaaatgcaaaaacccacacctctggaagataccttcacggccgctgctggagcttctgttgctgtgaatacttctctcagtgtgagaggttagccgtgatgaaagcagcgttacttctgaccgtgcctgagtaagagaatgctgatgccataactttatgtgtcgatacttgtcaaatcagttactgttcaggggatccttctgtttctcacggggtgaaacatgtctttagttcctcatgttaacacgaagccagagcccacatgaactgttggatgtcttccttagaaagggtaggcatggaaaattccacgaggctcattctcagtatctcattaactcattgaaagattccagttgtatttgtcacctggggtgacaagaccagacaggctttcccaggcctgggtatccagggaggctctgcagccctgctgaagggccctaactagagttctagagtttctgattctgtttctcagtagtccttttagaggcttgctatacttggtctgcttcaaggaggtcgaccttctaatgtatgaagaatgggatgcatttgatctcaagaccaaagacagatgtcagtgggctgctctggccctggtgtgcacggctgtggcagctgttgatgccagtgtcctctaactcatgctgtccttgtgattaaacacttctatctcccttgggaataagcacatacaggcttaagctctaagataggtgtttgtccttttaccatcgagctacttcccataataaccactttgcatccaacactcttcacccacctcccatacgcaaggggatgtggatacttggcccaaagtaactggtggtaggaatcttagaaacaagaccacttatactgtctgtctgaggcagaagataacagcagcatctcgaccagcctctgccttaaaggaaatctttattaatcacgtgtggttcacagataattctttttttaaaaaaacccaacctcctagagaagcacaactgtcaagagtggcttcagctttgcatcacgagtcttgtattccaagaaaatcaaagtggtacaatttgtttgtttacactatgatactttctaaataaactcttttttttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - protein, 285 aa, Homo sapiens
MPARPGRLLPLLARPAALTALLLLLLGHGGGGRWGARAQEAAAAAADGPPAADGEDGQDPHSKHLYTADMFTHGIQSAAHFVMFFAPWCGHCQRLQPTWNDLGDKYNSMEDAKVYVAKVDCTAHSDVCSAQGVRGYPTLKLFKPGQEAVKYQGPRDFQTLENWMLQTLNEEPVTPEPEVEPPVPPSSSKGCMSSQQATLSCTLHKATTLSSSSLRGVVTAKPWLQPGSSWLWALNIPKLSRLARLIVHSTMNSAPETRFVAIPLFSGSEMGKRWISTRESGIWSH

SEQ ID NO: 17 - DNA, 2133 bp, Homo sapiens
  CDS (185)...(1633)
ggatccaaagaattcggcacgaggagctacggttctggctgcgtcctagaggcatccggggcagtaaaaccgctgcgatcgcggaggcggcggccaggccgagaggcaggccgggcaggggtgtcggacgcagggcgctgggccgggtttcggcttcggccacagctttttttctcaaggtgcaatgaaagccttccacactttctgtgttgtccttctggtgtttgggagtgtctctgaagccaagtttgatgattttgaggatgaggaggacatagtagagtatgatgataatgacttcgctgaatttgaggatgtcatggaagactctgttactgaatctcctcaacgggtcataatcactgaagatgatgaagatgagaccactgtggagttggaagggcaggatgaaaaccaagaaggagattttgaagatgcagatacccaggagggagatactgagagtgaaccatatgatgatgaagaatttgaaggttatgaagacaaaccagatacttcttctagcaaaaataaagacccaataacgattgttgatgttcctgcacacctccagaacagctgggagagttattatctagaaattttgatggtgactggtctgcttgcttatatcatgaattacatcattgggaagaataaaaacagtcgccttgcacaggcctggtttaacactcatagggagcttttggagagcaactttactttagtgggggatgatggaactaacaaagaagccacaagcacaggaaagttgaaccaggagaatgagcacatctataacctgtggtgttctggtcgagtgtgctgtgagggcatgcttatccagctgaggttcctcaagagacaagacttactgaatgtcctggcccggatgatgaggccagtgagtgatcaagtgcaaataaaagtaaccatgaatgatgaagacatggatacctacgtatttgctgttggcacacggaaagccttggtgcgactacagaaagagatgcaggatttgagtgagttttgtagtgataaacctaagtctggagcaaagtatggactgccggactctttggccatcctgtcagagatgggagaagtcacagacggaatgatggatacaaagatggttcactttcttacacactatgctgacaagattgaatctgttcatttttcagaccagttctctggtccaaaaattatgcaagaggaaggtcagcctttaaagctacctgacactaagaggacactgttgtttacatttaatgtgcctggctcaggtaacacttacccaaaggatatggaggcactgctacccctgatgaacatggtgatttattctattgataaagccaaaaagttccgactcaacagagaaggcaaacaaaaagcagataagaaccgtgcccgagtagaagagaacttcttgaaactgacacatgtgcaaagacaggaagcagcacagtctcggcgggaggagaaaaaaagagcagagaaggagcgaatcatgaatgaggaagatcctgagaaacagcgcaggctggaggaggctgcattgaggcgtgagcaaaagaagttggaaaagaagcaaatgaaaatgaaacaaatcaaagtgaaagccatgtaaagccatcccagagatttgagttctgatgccacctgtaagctctgaattcacaggaaacatgaaaaacgccagtccatttctcaaccttaaatttcagacagtcttgggcaactgagaaatccttatttcatcatctactctgtttggggtttggggttttacagagattgaagatacctggaaagggctctgtttcaagaatttttttttccagataatcaaattattttgattattttataaaaggaatgatctatgaaatctgtgtaggttttaaatattttaaaaattataatacaaatcatcagtgcttttagtacttcagtgtttaaagaaataccatgaaatttataggtagataaccagattgttgctttttgtttaaaccaagcagttgaaatggctataaagactgactctaaaccaagattctgcaaataatgattggaattgcacaataaacattgcttgatgtttaaaaaaaaaaaaaaaaaaaa

SEQ ID NO: 18 - protein, 483 aa, Homo sapiens
MKAFHTFCVVLLVFGSVSEAKFDDFEDEEDIVEYDDNDFAEFEDVMEDSVTESPQRVIITEDDEDETTVELEGQDENQEGDFEDADTQEGDTESEPYDDEEFEGYEDKPDTSSSKNKDPITIVDVPAHLQNSWESYYLEILMVTGLLAYIMNYIIGKNKNSRLAQAWFNTHRELLESNFTLVGDDGTNKEATSTGKLNQENEHIYNLWCSGRVCCEGMLIQLRFLKRQDLLNVLARMMRPVSDQVQIKVTMNDEDMDTYVFAVGTRKALVRLQKEMQDLSEFCSDKPKSGAKYGLPDSLAILSEMGEVTDGMMDTKMVHFLTHYADKIESVHFSDQFSGPKIMQEEGQPLKLPDTKRTLLFTFNVPGSGNTYPKDMEALLPLMNMVIYSIDKAKKFRLNREGKQKADKNRARVEENFLKLTHVQRQEAAQSRREEKKRAEKERIMNEEDPEKQRRLEEAALRREQKKLEKKQMKMKQIKVKAM

SEQ ID NO: 19 - DNA, 1590 bp, Homo sapiens
  CDS (100)...(966)
ggatccaaagaattcggcacgagggtgggctggcgagccgacgcggcggcggaggaggctgtgaggagtgtgtggaacaggacccgggacagaggaaccatggctccgcagaacctgagcaccttttgcctgttgctgctatacctcatcggggcggtgattgccggacgagatttctataagatcttgggggtgcctcgaagtgcctctataaaggatattaaaaaggcctataggaaactagccctgcagcttcatcccgaccggaaccctgatgatccacaagcccaggagaaattccaggatctgggtgctgcttatgaggttctgtcagatagtgagaaacggaaacagtacgatacttatggtgaagaaggattaaaagatggtcatcagagctcccatggagacattttttcacacttctttggggattttggtttcatgtttggaggaacccctcgtcagcaagacagaaatattccaagaggaagtgatattattgtagatctagaagtcactttggaagaagtatatgcaggaaattttgtggaagtagttagaaacaaacctgtggcaaggcaggctcctggcaaacggaagtgcaattgtcggcaagagatgcggaccacccagctgggccctgggcgcttccaaatgacccaggaggtggtctgcgacgaatgccctaatgtcaaactagtgaatgaagaacgaacgctggaagtagaaatagagcctggggtgagagacggcatggagtacccctttattggagaaggtgagcctcacgtggatggggagcctggagatttacggttccgaatcaaagttgtcaagcacccaatatttgaaaggagaggagatgatttgtacacaaatgtgacaatctcattagttgagtcactggttggctttgagatggatattactcacttggatggtcaaggtacatatttcccgggataagatcaccaggccaggagcgaagctatggaagaaaggggaagggctccccaactttgacaacaacaatatcaagggctctttgataatcacttttgatgtggattttccaaaagaacagttaacagaggaagcgagagaaggtatcaaacagctactgaaacaagggtcagtgcagaaggtatacaatggactgcaaggatattgagagtgaataaaattggactttgtttaaaataagtgaataagcgatatttattatctgcaaggtttttttgtgtgtgtttttgtttttattttcaatatgcaagttaggcttaatttttttatctaatgatcatcatgaaatgaataagagggcttaagaatttgtccatttgcattcggaaaagaatgaccagcaaaaggtttactaatacctctccctttggggatttaatgtctggtgctgccgcctgagtttcaagaattaaagctgcaagaggactccaggagcaaaagaaacccaatatagagggttggagttgttagcaatttcattcaaaatgccaactggagaagtctgtttttaaatacattttgttgttatttttaaaaaaaaaaaaaaaaaaaaaaaaaaaaaaa

SEQ ID NO: 20 - protein, 289 aa, Homo sapiens
MAPQNLSTFCLLLLYLIGAVIAGRDFYKILGVPRSASIKDIKKAYRKLALQLHPDRNPDDPQAQEKFQDLGAAYEVLSDSEKRKQYDTYGEEGLKDGHQSSHGDIFSHFFGDFGFMFGGTPRQQDRNIPRGSDIIVDLEVTLEEVYAGNFVEVVRNKPVARQAPGKRKCNCRQEMRTTQLGPGRFQMTQEVVCDECPNVKLVNEERTLEVEIEPGVRDGMEYPFIGEGEPHVDGEPGDLRFRIKVVKHPIFERRGDDLYTNVTISLVESLVGFEMDITHLDGQGTYFPG

SEQ ID NO: 21 - DNA, 1994 bp, Homo sapiens
  CDS (132)...(1886)
gagagcagaccaggcccggtggagaattaggtgttgttgggagctcctgcctcccacaggattccagctgcagggagcctcagggactctgggccgcacggagttgggggcattccccagagagcgtcgccatggtctgcagggagcagttatcaaagaatcaggtcaagtgggtgtttgccggcattacctgtgtgtctgtggtggtcattgccgcaatagtccttgccatcaccctgcggcggccaggctgtgagctggaggcctgcagccctgatgccgacatgctggactacctgccgagcctgggccagatcagccagcgagatgccttggaggtcacctggtaccacgcagccaacagcaacaaagccatgacagctgccctgaacagcaacatcacagtcctggaggctgacgtcaatgtagaagggctcggcacagccaatgagacaggagttcccatcatggcacacccccccactatctacagtgacaacacactggagcagtggctggacgctgtgctgggctcttcccaaaagggcatcaaactggacttcaagaacatcaaggcagtgggcccctccctggacctcctgcggcagctgacagaggaaggcaaagtccggcggcccatatggatcaacgctgacatcttaaagggccccaacatgctcatctcaactgaggtcaatgccacacagttcctggccctggtccaggagaagtatcccaaggctaccctatctccaggctggaccaccttctacatgtccacgtccccaaacaggacgtacacccaagccatggtggagaagatgcacgagctggtgggaggagtgccccagagggtcaccttccctgtacggtcttccatggtgcgggctgcctggccccacttcagctggctgctgagccaatctgagaggtacagcctgacgctgtggcaggctgcctcggaccccatgtcggtggaagatctgctctacgtccgggataacactgctgtccaccaagtctactatgacatctttgagcctctcctgtcacagttcaagcagctggccttgaatgccacacggaaaccaatgtactacacgggaggcagcctgatccctcttctccagctgcctggggatgacggtctgaatgtggagtggctggttcctgacgtccagggcagcggtaaaacagcaacaatgaccctcccagacacagaaggcatgatcctgctgaacactggcctcgagggaacagtggctgaaaaccccgtgcccattgttcatactccaagtggcaacatcctgacgctggagtcctgcctgcagcagctggccacacatcccgggcactggggcatccatttgcaaatagcggagcccgcagccctccggccatccctggccttgctggcacgcctctccagccttggcctcttgcattggcctgtgtgggttggggccaaaatctcccacgggagttttttggtccccggccatgtggctggcagagagctgcttacagctgtggctgaggtcttcccccacgtgactgtggcaccaggctggcctgaggaggtgctgggcagtggctacagggaacagctgctcacagatatgctagagttgtgccaggggctctggcaacctgtgtccttccagatgcaggccatgctgctgggccacagcacagctggagccatagccaggctgctggcatcctccccccgggccaccgtcacagtggagcacaacccagctgggggcgactatgcctctgtgaggacagcattgctggcagctagggctgtggacaggacccgagtctactacaggctaccccagggctaccacaaggacttgctggctcatgttggtagaaactgagcacccaggggtggtgggccagcggacctcagggcggaggcttcccacggggaggcaggaagaaataaaggtctttggcttacaaaaaaaaaaaacaaaaaaaaa

SEQ ID NO: 22 - protein, 585 aa, Homo sapiens
MVCREQLSKNQVKWVFAGITCVSVVVIAAIVLAITLRRPGCELEACSPDADMLDYLPSLGQISQRDALEVTWYHAANSNKAMTAALNSNITVLEADVNVEGLGTANETGVPIMAHPPTIYSDNTLEQWLDAVLGSSQKGIKLDFKNIKAVGPSLDLLRQLTEEGKVRRPIWINADILKGPNMLISTEVNATQFLALVQEKYPKATLSPGWTTFYMSTSPNRTYTQAMVEKMHELVGGVPQRVTFPVRSSMVRAAWPHFSWLLSQSERYSLTLWQAASDPMSVEDLLYVRDNTAVHQVYYDIFEPLLSQFKQLALNATRKPMYYTGGSLIPLLQLPGDDGLNVEWLVPDVQGSGKTATMTLPDTEGMILLNTGLEGTVAENPVPIVHTPSGNILTLESCLQQLATHPGHWGIHLQIAEPAALRPSLALLARLSSLGLLHWPVWVGAKISHGSFLVPGHVAGRELLTAVAEVFPHVTVAPGWPEEVLGSGYREQLLTDMLELCQGLWQPVSFQMQAMLLGHSTAGAIARLLASSPRATVTVEHNPAGGDYASVRTALLAARAVDRTRVYYRLPQGYHKDLLAHVGRN

SEQ ID NO: 23 - DNA, 1340 bp, Homo sapiens
  CDS (79)...(918)
ggcgcttgcgctgagcagcccgcgagggcggaagtgggagctgcgaccgcgctccctgtgaggtgggcaagcggcgaaatggcgccctccgggagtcttgcagttcccctggcagtcctggtgctgttgctttggggtgctccctggacgcacgggcggcggagcaacgttcgcgtcatcacggacgagaactggagagaactgctggaaggagactggatgatagaattttatgccccgtggtgccctgcttgtcaaaatcttcaaccggaatgggaaagttttgctgaatggggagaagatcttgaggttaatattgcgaaagtagatgtcacagagcagccaggactgagtggacggtttatcataactgctcttcctactatttatcattgtaaagatggtgaatttaggcgctatctgggtccaaggactaagaaggacttcataaactttataagtgataaagagtggaagagtattgagcccgtttcatcatggtttggtccaggttctgttctgatgagtagtatgtcagcactctttcagctatctatgtggatcaggacgtgccataactactttattgaagaccttggattgccagtgtggggatcatatactgtttttgctttagcaactctgttttccggactgttattaggactctgtatgatatttgtggcagattgcctttgtccttcaaaaaggcgcagaccacagccatacccatacccttcaaaaaaattattatcagaatctgcacaacctttgaaaaaagtggaggaggaacaagaggcggatgaagaagatgtttcagaagaagaagctgaaagtaaagaaggaacaaacaaagactttccacagaatgccataagacaacgctctctgggtccatcattggccacagataaatcctagttaaattttatagttatcttaatattatgattttgataaaaacagaagattgatcattttgtttggtttgaagtgaactgtgacttttttgaatattgcagggttcagtctagattgtcattaaattgaagagtctacattcagaacataaaagcactaggtatacaagtttgaaatatgatttaagcacagtatgatggtttaaatagttctctaatttttgaaaaatcgtgccaagcaataagatttatgtgtatttgtttaataataacctatttcaagtctgagttttgaaaatttacatttcccaagtattgcattattgaggtatttaagaagattattttagagaaaaatatttctcatttgatataatttttctctgtttcactgtgaaaaaaaaaaaaaaaaaaaaaaaa

SEQ ID NO: 24 - protein, 280 aa, Homo sapiens
MAPSGSLAVPLAVLVLLLWGAPWTHGRRSNVRVITDENWRELLEGDWMIEFYAPWCPACQNLQPEWESFAEWGEDLEVNIAKVDVTEQPGLSGRFIITALPTIYHCKDGEFRRYLGPRTKKDFINFISDKEWKSIEPVSSWFGPGSVLMSSMSALFQLSMWIRTCHNYFIEDLGLPVWGSYTVFALATLFSGLLLGLCMIFVADCLCPSKRRRPQPYPYPSKKLLSESAQPLKKVEEEQEADEEDVSEEEAESKEGTNKDFPQNAIRQRSLGPSLATDKS

SEQ ID NO: 25 - DNA, 1011 bp, Homo sapiens
  CDS (128)...(985)
ggagactttaacatcagaaaaaggatggacttgttgcagttgctgtagcattcaaagtcaaggtgatcatttcaaaccaagcatcagcaacaattaaaaatattcacttggtatctgtagtttaataatggaccaacatcaacatttgaataaaacagcagagtcagcatcttcagagaaaaagaaaacaagacgctgcaatggattcaagatgttcttggcagccctgtcattcagctatattgctaaagcactaggtggaatcattatgaaaatttccatcactcaaatagaaaggagatttgacatatcctcttctcttgctggtttaattgatgtaagctttgaaattggaaatttgcttgtgattgtatttgtaagttactttggatctaaactacacagaccgaagttaattggaattggttgtctccttatgggaactggaagtattttgacagctttaccacatttcttcatgggatattataggtattctaaagaaacccatattaatccatcagaaaattcaacatcaagtttatcaacctgtttaattaatcaaaccttaccattcaatggaacatcacctgagatagtagaaaaagattgtgtaaaggaatctgggtcacacatgtggatctatgtcttcatggggaatatgcttcgtggcataggggaaacccccatagtaccattggggatttcatacattgatgattttgcaaaagaaggacattcttccttgtatttaggtagtttgaatgcaataggaatgattggtccagtcattggctttgcactgggatctctgtttgctaaaatatacgtggatattggatatgtagatctgagcactatcagaataactcctaaggactctcgttgggttggagcttggtggcttggtttccttgtgtctggactattttccattatttcttccataccattttttttcttgccgaaaaatccaaataaaccataaaaaaaaaaaaaaaaaaaaaaaaa

SEQ ID NO: 26 - protein, 286 aa, Homo sapiens
MDQHQHLNKTAESASSEKKKTRRCNGFKMFLAALSFSYIAKALGGIIMKISITQIERRFDISSSLAGLIDVSFEIGNLLVIVFVSYFGSKLHRPKLIGIGCLLMGTGSILTALPHFFMGYYRYSKETHINPSENSTSSLSTCLINQTLPFNGTSPEIVEKDCVKESGSHMWIYVFMGNMLRGIGETPIVPLGISYIDDFAKEGHSSLYLGSLNAIGMIGPVIGFALGSLFAKIYVDIGYVDLSTIRITPKDSRWVGAWWLGFLVSGLFSIISSIPFFFLPKNPNKP

SEQ ID NO: 27 - DNA, 2027 bp, Homo sapiens
  CDS (270)...(1286)
gacatttcattgtaaacgactgggagtatctgagcaaattatttcttagctgactttagagaaaacggctacctatctgaccccaaaacgacttgaggaaactgtttccacggtcctgctgcagaggggaagcacagtcgtcaagaagagagtggggtcaggatcaaaacacatttagtgtgacttagggaaagaaaacattttccctctttgaacctctctggatacagtcattttgcctctacttgaggatcaactgttcaacctcaatggcctttcaggacctcctgggtcacgctggtgacctgtggagattccagatccttcagactgtttttctctcaatctttgctgttgctacataccttcattttatgctggagaacttcactgcattcatacctggccatcgctgctgggtccacatcctggacaatgacactgtctctgacaatgacactggggccctcagccaagatgcactcttgagaatctccaccccactggactcaaacatgaggccagagaagtgtcgtcgctttgttcatcctcagtggcagctccttcacctgaatgggaccttccccaacacaagtgacgcagacatggagccctgtgtggatggctgggtgtatgacagaatctccttctcatccgccatcgtgactgagtgggatctggtatgtgactctcaatcactgacttcagtggctaaatttgtattcatggctggaatgatggtgggaggcatcctaggcggtcatttatcagacaggtttgggagaaggttcgtgctcagatggtgttacctccaggttgccattgttggcacctgtgcagccttggctcccaccttcctcatttactgcttactacgcttcttgtctgggattgctgcaatgagcctcataacaaatactattatgttaatagccgagtgggcaacacacagattccaggccatgggaattacattgggaatgtgcccttctggtattgcatttatgaccctggcaggcctggcttttgccattcgagactggcatatcctccagctggtggtgtctgtaccatactttgtgatctttctgacctcaagttggctgctagagtctgctcggtggctcattatcaacaataaaccagaggaaggcttaaaggaacttagaaaagctgcacacaggagtggaatgaagaatgccagagacaccctaaccctggagattttgaaatccaccatgaaaaaagaactggaggcagcacaaaaaaaaaaaaccttctctgtgtgaaatgctccacatgcccaacatatgtaaaaggatctccctcctgtcctttacgagatttgcaaactttatggcctattttggccttaatctccatgtccagcatctggggaacaatgttttcctgttgcagactctctttggtgcagtcatcctcctggccaactgtgttgcaccttgggcactgaaatacatgaaccgtcgagcaagccagatgcttctcatgttcctactggcaatctgccttctggccatcatatttgtgccacaagaaatgcagacgctgcgtgaggttttggcaacactgggcttaggagcgtctgctcttgccaatacccttgcttttgcccatggaaatgaagtaattcccaccataatcagggcaagagctatggggatcaatgcaacctttgctaatatagcaggagccctggctcccctcatgatgatcctaagtgtgtattctccacccctgccctggatcatctatggagtcttccccttcatctctggctttgctttcctcctccttcctgaaaccaggaacaagcctctgtttgacaccatccaggatgagaaaaatgagagaaaagaccccagagaaccaaagcaagaggatccgagagtggaagtgacgcagttttaaggaattccaggagctgactgccgatcaatgagccagatgaagggaacaatcaggactattcctagacactagcaaaaaaaaaaaaaaaaaaaaaa

SEQ ID NO: 28 - protein, 339 aa, Homo sapiens
MAFQDLLGHAGDLWRFQILQTVFLSIFAVATYLHFMLENFTAFIPGHRCWVHILDNDTVSDNDTGALSQDALLRISTPLDSNMRPEKCRRFVHPQWQLLHLNGTFPNTSDADMEPCVDGWVYDRISFSSAIVTEWDLVCDSQSLTSVAKFVFMAGMMVGGILGGHLSDRFGRRFVLRWCYLQVAIVGTCAALAPTFLIYCLLRFLSGIAAMSLITNTIMLIAEWATHRFQAMGITLGMCPSGIAFMTLAGLAFAIRDWHILQLVVSVPYFVIFLTSSWLLESARWLIINNKPEEGLKELRKAAHRSGMKNARDTLTLEILKSTMKKELEAAQKKKTFSV

SEQ ID NO: 29 - DNA, 2389 bp, Homo sapiens
  CDS (204)...(1244)
gggaaacaggaagtcgcccatcctcctcgcccggcggcagctgtccccgagcgggaggagcccgagggggcgcgagccccgcatgaatcattgtagtcaatcattttccagttctcagccgctcagttgtgatcaagggacacgtggtttccgaactgccagctcagaataggaaaataacttgggattttatattggaagacatggatcttgctgccaacgagatcagcatttatgacaaactttcagagactgttgatttggtgagacagaccggccatcagtgtggcatgtcagagaaggcaattgaaaaatttatcagacagctgctggaaaagaatgaacctcagagaccccccccgcagtatcctctccttatagttgtgtataaggttctcgcaaccttgggattaatcttgctcactgcctactttgtgattcaacctttcagcccattagcacctgagccagtgctttctggagctcacacctggcgctcactcatccatcacattaggctgatgtccttgcccattgccaagaagtacatgtcagaaaataagggagttcctctgcatgggggtgatgaagacagaccctttccagactttgacccctggtggacagacgactgtgagcagaatgagtcagagcccattcctgccaactgcactggctgtgcccagaaacacctgaaggtgatgctcctggaagacgccccaaggaaatttgagaggctccatccactggtgatcaagacgggaaagcccctgttggaggaggagattcagcattttttgtgccagtaccctgaggcgacagaaggcttctctgaagggtttttcgccaagtggtggcgctgctttcctgagcggtggttcccatttccttatccatggaggagacctctgaacagatcacaaatgttacgtgagctttttcctgttttcactcacctgccatttccaaaagatgcctctttaaacaagtgctcctttcttcacccagaacctgttgtggggagtaagatgcataagatgcctgacctatttatcattggcagcggtgaggccatgttgcagctcatccctcccttccagtgccgaagacattgtcagtctgtggccatgccaatagagccaggggatatcggctatgtcgacactacccactggaaggtctacgttatagccagaggggtccagcctttggtcatctgcgatggaaccgctttctcagaactgtaggaaatagaactgtgcacaggaacagcttccagagccgaaaaccaggttgaaaggggaaaaataaaaacaaaaacgatgaaactgctttctgggggttggttacttagttacctgccctttgcatgcatgtgtgaaccagctgtgagctgcagggcagtggccagagcctcgccctcctgactcttcctgcaggtggctcaggaaggattcagcctggccacttggctaggactctgccagcacccatctgagactgacctcttccgggcctttggacactatgaccttgatgctgcccttcaggcaggaaacagggctggtgcctttcttcacctgcatggccagcttccttccctggcagtggagagggcagccaacaggttctaatgtcagagccatcctttaccaggtgggcctgcttgtccccgtcttgcctgccacatcactctactttttggaaggccatggctgattaaagaagttcttgtagtttcccaagcaaagtggaatctagaaacagtgaaaaaagttcagataactttgaattgcattcaagaagtacacttctttcccattgtccgtggctcttggagtctccgtgatgccaggctagagtctgattatataataattcaaaatggtaactcccaaggtaatgctttcttccatttcatcaggttcttttatccccactgcaccccctccccttctcccttgcctatctggatggcttctcagaagctcggccctagtcctccctgccttggcggggccagagcccactactgctgaggcagcactgctctcgtcagctgtgttgcctttaccaagtgtcttcagagggttatgagttagagtagctggcctggggagagggtgcctccctgggtttgatctttagggtctgactttctgcagagaagatgttttacagatgtgtcaaagctgatgtaatgtggttgggggaggaaatccagacccaaagtgtttgtcagctgggtgtgcaactgcctatgtgatcctctgtcttaaaatgatttctgtctgtgctgcgaaacaaagacaaggtgaggtgtttttcttttttgtaataatataaagctgtgtgtttcaaaaaaaaaaaaaaaaaaaaaaaaaaaaaa

SEQ ID NO: 30 - protein, 347 aa, Homo sapiens
MDLAANEISIYDKLSETVDLVRQTGHQCGMSEKAIEKFIRQLLEKNEPQRPPPQYPLLIVVYKVLATLGLILLTAYFVIQPFSPLAPEPVLSGAHTWRSLIHHIRLMSLPIAKKYMSENKGVPLHGGDEDRPFPDFDPWWTDDCEQNESEPIPANCTGCAQKHLKVMLLEDAPRKFERLHPLVIKTGKPLLEEEIQHFLCQYPEATEGFSEGFFAKWWRCFPERWFPFPYPWRRPLNRSQMLRELFPVFTHLPFPKDASLNKCSFLHPEPVVGSKMHKMPDLFIIGSGEAMLQLIPPFQCRRHCQSVAMPIEPGDIGYVDTTHWKVYVIARGVQPLVICDGTAFSEL